(19) United States
(12) Patent Application Publication
(10) Pub. No.: US 2021/0228853 A1
(43) Pub. Date: Jul. 29, 2021

(54) DRUG DELIVERY METHODS TARGETING THE LYMPHATIC SYSTEM

(71) Applicant: Sorrento Therapeutics, Inc., San Diego, CA (US)

(72) Inventor: RUSSELL FREDERICK ROSS, San Diego, CA (US)

(21) Appl. No.: 15/733,817

(22) PCT Filed: May 30, 2019
(86) PCT No.: PCT/US2019/034736

§ 371 Date: Nov. 30, 2020

Related U.S. Application Data
(60) Provisional application No. 62/678,584, filed on May 31, 2018.
(60) Provisional application No. 62/678,592, filed on May 31, 2018.
(60) Provisional application No. 62/678,601, filed on May 31, 2018.

(51) Int. Cl. A61M 37/00; A61K 9/00; C07K 16/28; A61K 38/17; A61K 47/68; A61K 47/64; C07K 16/24; A61P 35/04; A61P 19/02

(52) U.S. Cl. CPC A61M 37/0015; A61K 9/0021; C07K 16/2818; A61K 38/1793; A61K 47/6811; A61M 2037/0061; C07K 16/241; A61P 35/04; A61P 19/02; A61M 2037/0023; A61K 47/6425

(57) ABSTRACT

Disclosed herein is a method for administering a therapeutic agent to multiple regions of the lymphatic system of a patient. The method generally includes placing two medical devices comprising a plurality of microneedles on the skin of the patient at two different locations proximate lymph vessels and/or lymph capillaries that drain into the right lymphatic duct and the thoracic duct; inserting the plurality of microneedles of medical devices into the patient to a depth whereby at least the epidermis is penetrated and administering via the microneedles of the medical devices a therapeutic agent into the lymphatic system of the patient. Disclosed herein also is a method for preventing or reducing cancer metastasis in a patient. Disclosed herein also is a method for treating an inflammatory medical condition in a patient.

This application claims the benefit of priority of U.S. Provisional Patent Application No. 62/678,601, filed May 31, 2018; U.S. Provisional Patent Application No. 62/678,592, filed May 31, 2018; and U.S. Provisional Patent Application No. 62/678,584, filed May 31, 2018.

### INTRODUCTION AND SUMMARY

The field of the disclosure relates generally to the administration of a medicament to the lymphatic system of a patient by use of a fluid delivery apparatus that enables the targeting of specific lymph nodes. More specifically, this disclosure relates to the administration of a medicament to a patient suffering from medical condition that can be ameliorated by the administration of a medicament to the lymphatic system of the patient.

The lymphatic system plays an important role in transporting body fluids and particulate materials throughout the body. The lymphatic system comprises several lymph organs (e.g., the spleen and thymus) in addition to lymph nodes, lymph vessels and lymph capillaries. The vessels transport lymph fluid around the body in a single direction in either the superficial vessels or the deep vessels (i.e., the lymphatic vasculature). Drainage begins in blind capillaries which gradually develop into vessels. These vessels then travel through several lymph nodes. The lymph nodes contain both T and B lymphocytes in addition to other cells associated with the immune system. Antigens and other foreign particles are filtered out in the lymph nodes. The lymph vessels eventually end in either the right lymphatic duct which drains into the right internal jugular vein or the thoracic duct which drains into the subclavian vein. It is a one-way system where the lymph fluid (also referred to a lymph) is eventually returned to the circulatory system of the patient.

Large proteins and certain cells (lymphocytes) pass from the blood plasma into the tissue fluid, and it is an important function of the lymph (i.e., the fluid in the lymphatic system) to return these essential components to the blood circulation. The lymph also plays an important role in transporting the products of fat digestion in the gastrointestinal tract, the chylomicrons, and into the blood circulation.

Numerous devices have been developed for transdermal drug delivery using microneedle assemblies or arrays. Microneedle assemblies reduce the amount of pain felt by a patient as compared to larger conventional needles. Moreover, conventional subcutaneous (and often intra-muscular) delivery of medicines using a needle operates to deliver a large quantity of the medicine at one time, thereby creating a spike in the bioavailability of the therapeutic agent. While this is not a significant problem for some medicaments, many medical conditions benefit from having a steady state concentration of the active therapeutic agent for an extended period of time. Transdermal delivery apparatus are capable of administering medicaments at a substantially constant rate over an extended period of time. Some devices are capable of delivering a medicament directly into the lymphatic system of a patient. One such device is the Sofusa™ drug delivery platform available from Sorrento Therapeutics, Inc.

Metastasis is thought to be directly or indirectly responsible for more than 90% of all cancer deaths, and the lymphatic system plays a significant role in cancer metastasis. Malignant cells may enter the lymphatic system and are captured by lymph nodes where secondary tumors can be produced. Eventually the whole of the lymph chain can become involved. The lymphatic system is also often involved in the spread of tumors to other parts of the body (i.e., metastasis). Consequently, there is need for a method of preventing or reducing the spread of malignant cells via the lymphatic system. Increased lymphatic density is often associated with malignant tumors due to the induction of lymphangiogenesis. This increases the chance that invasive cancer cells will enter the lymphatic system which in turn leads to tumor dissemination from the regional lymph nodes throughout the patient and poor patient outcomes. Because the lymph nodes are frequently the first stop of spreading cancer cells, being able to selectively target and treat these cells is important when considering the most effective treatment for a patient. Thus, there is a need to be able to selectively deliver a medicament to specific lymph nodes in order to treat and/or kill malignant cells.

Tumor necrosis factor alpha (TNF-α) has become a significant therapeutic target in connection with a large variety of medical conditions, including rheumatoid arthritis (RA), juvenile arthritis, psoriatic arthritis, plaque psoriasis, ankylosing spondylitis, ulcerative colitis (UC), and Crohn's disease. Multiple drugs that specifically target TNF-α have received FDA approval including Adalimumab (Humira®), Adalimumab-atto (Amjevita®, a biosimilar to Humira®), Certolizumab pegol (Cimzia®), etanercept (Enbrel®), etanercept-szzs (Ereizi®, a biosimilar to Enbrel®), Golimumab (Simponi®, Simponi Aria®), Infliximab (Remicade®), and Infliximab-dyyb (Inflectra®, a biosimilar to Remicade®), while literally dozens of clinical trials are ongoing with either new therapeutic agents or expanded uses for currently approved ones. Known side effects for TNF-α inhibitors include headaches, heartburn, nausea, vomiting, allergic reactions and muscle weakness. Because TNF-α plays an important role in the immune system, altering TNF-α activity makes a patient more susceptible to secondary infections or some cancers. As such, there is need to develop a dosing regimen or method that maintains a therapeutically effective dose of the therapeutic agent in a patient while reducing the overall patient exposure to the therapeutic agent.

Accordingly, the following embodiments are provided.

Embodiment 1 is a method for administering a therapeutic agent to the lymphatic system of a patient, the method comprising:

placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography;
placing a second medical device comprising a plurality of microneedles on the skin of the patient at a second location proximate to a second position under the skin of the patient, optionally wherein the first and second medical devices are the same device, wherein the second position is proximate to lymph vessels and/or lymph capillaries that drain into the thoracic duct, and wherein the microneedles of the second medical device have a surface comprising nanotopography;
inserting the plurality of microneedles of the first medical device into the patient to a depth whereby at least the epidermis is penetrated and an end of at least one of the microneedles is proximate to the first position;
inserting the plurality of microneedles of the second medical device into the patient to a depth whereby at least the epidermis is penetrated and an end of at least one of the microneedles is proximate to the second position; and
administering via the microneedles of the first medical device a first dose of the therapeutic agent into the first position; and administering via the microneedles of the second medical device a second dose of the therapeutic agent into a second position.

Embodiment 2 is the method according to embodiment 1, wherein administering the first dose and administering the second dose is simultaneous.

Embodiment 3 is the method according to any one of embodiment 1 or 2, wherein administering the first dose and administering the second dose partially overlap in time.

Embodiment 4 is the method according to any one of embodiments 1-3, wherein administering the first dose and administering the second dose is sequential.

Embodiment 5 is the method according to any one of embodiments 1-4, wherein the first and second medical devices are different devices.

Embodiment 6 is the method according to embodiment 4, wherein the first and second medical devices are the same device.

Embodiment 7 is the method according to any one of embodiments 1-6, wherein administering the doses cumulatively provides a therapeutically effective amount of the therapeutic agent.

Embodiment 8 is the method according to any one of embodiments 1-7, wherein the first location and the second location are on different limbs of the patient.

Embodiment 9 is the method according to any one of embodiments 1-8, wherein the first location and the second location are each independently proximate to the hands or the feet of the patient.

Embodiment 10 is the method according to any one of embodiments 1-9, wherein one of the first location or the second location is on the right arm or the right leg of the patient and the other location is on the left arm or the left leg of the patient.

Embodiment 11 is the method according to any one of embodiments 1-10, wherein the method further comprises:

a. placing a third medical device comprising a plurality of microneedles on the skin of the patient at a third location proximate to a third position under the skin of the patient, wherein the third position is proximate to lymph vessels and/or lymph capillaries;
b. inserting the plurality of microneedles of the third medical device into the patient to a depth whereby at least the epidermis is penetrated and an end of at least one of the microneedles is proximate to the third position; and
c. administering via the third medical device a third dose of said therapeutic agent; and
d. wherein the third location is different than the first location and the second location, and
e. the third position is different that the first position and the second position.

Embodiment 12 is the method according to embodiment 11 wherein the first location, the second location and the third location are on different limbs of the patient.

Embodiment 13 is the method according to embodiment 11 or 12, wherein the first position, the second position and the third position are selected such that the therapeutic agent is administered to regions of the lymphatic system that initially drain into different lymph nodes, and

wherein the draining lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes.

Embodiment 14 is the method according to any of embodiments 11-13, wherein the method further comprises:

a. placing a fourth medical device comprising a plurality of microneedles on the skin of the patient at a fourth location proximate to a fourth position under the skin of the patient, wherein the fourth position is proximate to lymph vessels and/or lymph capillaries;
b. inserting the plurality of microneedles of the fourth medical device into the patient to a depth whereby at least the epidermis is penetrated and an end of at least one of the microneedles is proximate to the fourth position; and
c. administering via the fourth medical device a fourth dose of said therapeutic agent; and
d. wherein the first location, the second location, the third location, and the fourth location are on different limbs of the patient.

Embodiment 15 is the method according to embodiment 14, wherein the first dose and the second dose are administered simultaneously, and the third dose and the fourth dose are administered simultaneously, and a beginning time for administering the first dose and the second dose is different than a beginning time for administering the third dose and the fourth dose with a period of time between the beginning times for administrating the doses.

Embodiment 16 is the method according to any of embodiment 14 or 15, wherein

a. the third position drains into the right lymphatic duct; and

b. the fourth position drains into the thoracic duct.

Embodiment 17 is the method of embodiment 16 wherein the first location and the third location on the skin of the patient are different from each other, and the first position and the third position are different from each other, and the first position and the third position are selected such that the therapeutic agent is administered to regions of the lymphatic system that initially drain into different lymph nodes.

Embodiment 18 is the method of embodiment 17, wherein the second location and the fourth location on the skin of the patient are different from each other, and the second portion and the fourth portion of the lymphatic system are different from each other, and the second position and the fourth position are selected such that the therapeutic agent is administered to regions of the lymphatic system that initially drain into different lymph nodes.

Embodiment 19 is the method according to any one of embodiments 1-18, wherein administering the first dose of the therapeutic agent and administering the second dose of the therapeutic agent partially overlap in time.

Embodiment 20 is a method for administering a therapeutic agent to the lymphatic system of a patient, the method comprising:

placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography;
placing a second medical device comprising a plurality of microneedles on the skin of the patient at a second location proximate to a second position under the skin of the patient, wherein the second position is proximate to lymph vessels and/or lymph capillaries that drain into the thoracic duct, and wherein the microneedles of the second medical device have a surface comprising nanotopography, optionally wherein the first and second medical devices are the same medical device;
inserting the plurality of microneedles of the first medical device into the patient to a depth whereby at least the epidermis is penetrated and an end of at least one of the microneedles is proximate to the first position;
inserting the plurality of microneedles of the second medical device into the patient to a depth whereby at least the epidermis is penetrated and an end of at least one of the microneedles is proximate to the second position;
administering via the microneedles of the first medical device a first dose of the therapeutic agent into the first position; and
administering via the microneedles of the second medical device a second dose of the therapeutic agent into the second position,
wherein a beginning time for administering the first dose and the second dose are different and separated by a period of time.

Embodiment 21 is the method according to embodiment 20, wherein the period of time is at least 4, 6, 8, 10, 12, 16, 24, 36, 48 or 72 hours.

Embodiment 22 is the method according to any one of embodiment 20 or 21, wherein the first dose, the second dose, or the first and second doses together constitute a therapeutically effective dose.

Embodiment 23 is the method according to any one of embodiments 20-22, wherein the first dose and the second dose are therapeutically effective doses.

Embodiment 24 is the method according to embodiments 20-23, wherein the method further comprises:

a. placing a third medical device comprising a plurality of microneedles on the skin of the patient at a third location proximate to a third position under the skin of the patient, wherein the third position is proximate to lymph vessels and/or lymph capillaries, and wherein the microneedles of the third medical device have a surface comprising nanotopography;
b. inserting the plurality of microneedles of the third medical device into the patient to a depth whereby at least the epidermis is penetrated and an end of at least one of the microneedles is proximate to the third position; and
c. administering via the microneedles of the third medical device a third dose of the therapeutic agent into the third position;
d. wherein the beginning time for administering the first dose, the second dose, and the third dose are each separated by a period of time; and
e. the first location, the second location, and the third location are located on different limbs of the patient.

Embodiment 25 is the method according to embodiments 20-23, wherein the method further comprises:

a. placing a third medical device comprising a plurality of microneedles on the skin of the patient at a third location proximate to a third position under the skin of the patient, wherein the third position is proximate to lymph vessels and/or lymph capillaries, and wherein the microneedles of the third medical device have a surface comprising nanotopography;
b. inserting the plurality of microneedles of the third medical device into the patient to a depth whereby at least the epidermis is penetrated and an end of at least one of the microneedles is proximate to the third position; and
c. administering via the microneedles of the third medical device a third dose of the therapeutic agent into the third position;
d. wherein the beginning time for administering the first dose, the second dose, and the effective dose are each separated by a period of time; and
e. wherein the first location and the third location are different and are selected such that the therapeutic agent is administered to regions of the lymphatic system that initially drain into different lymph nodes.

Embodiment 26 is the method according to any one of embodiments 24 or 25, wherein the first dose, the second dose, the third dose, or a combination of any two or more thereof constitute a therapeutically effective dose.

Embodiment 27 is the method according to any one of embodiments 24-26, wherein the first dose, the second dose, and the third dose are therapeutically effective doses.

Embodiment 28 is the method according to any of embodiments 24-27, wherein the first position and the third position flow initially into different lymph nodes.

Embodiment 29 is the method according to any of embodiments 24-28, wherein the method further comprises:

a. placing a fourth medical device comprising a plurality of microneedles on the skin of the patient at a fourth location proximate to a fourth position under the skin of the patient, wherein the fourth position is proximate to lymph vessels and/or lymph capillaries, and wherein the microneedles of the fourth medical device have a surface comprising nanotopography;
b. inserting the plurality of microneedles of the fourth medical device into the patient to a depth whereby at least the epidermis is penetrated and an end of at least one of the microneedles is proximate to the fourth position; and
c. administering via the microneedles of the fourth medical device a fourth therapeutically effective dose of the therapeutic agent into the fourth position; and
d. wherein the beginning time for administering the first dose, the second dose, the third dose, and the fourth dose are each separated by a period of time; and
e. wherein the lymph vessels and/or lymph capillaries of the third position drain into right lymphatic duct, and the lymph vessels and/or lymph capillaries of the fourth position drains into the thoracic duct,
f. wherein the first location and the third location are different and are selected such that the therapeutic agent is administered to regions of the lymphatic system that initially drain into different lymph nodes, and
g. wherein the second location and the fourth location are different and selected such that the therapeutic agent is administered to regions of the lymphatic system that initially drain into different lymph nodes.

Embodiment 30 is the method according to embodiment 29, wherein the first dose, the second dose, the third dose, the fourth dose, or a combination of any two or more thereof constitute a therapeutically effective dose.

Embodiment 31 is the method according to embodiment 29 or 30, wherein the first dose, the second dose, the third dose, and the fourth dose are therapeutically effective doses.

Embodiment 32 is the method according to any one of embodiment 1-31, wherein the first and second locations deliver to lymphatic capillaries and/or vessels that drain into different lymph nodes.

Embodiment 33 is the method according to any one of embodiments 1-32, wherein the lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

Embodiment 34 is the method of any one of embodiments 1-33, wherein the first location is a first arm and the second location is selected from a leg or arm on the opposite of the body of the patient.

Embodiment 35 is the method of any one of embodiments 1-34, wherein the therapeutic agent is an immune-suppressing agent.

Embodiment 36 is the method of any one of embodiments 1-35, wherein the therapeutic agent is adalimumab, adalimumab-atto, certolizumab pegol, etanercept, etanercept-szzs, golimumab, infliximab, infliximab-dyyb, ustekinumab, rituximab, secukinumab, omalizumab, natalizumab, ixekizumab, obinutuzumab, rituximab/hyaluronidase human, dor a biosimilar or bioequivalent of any of the foregoing.

Embodiment 37 is the method of any one of embodiments 1-36, wherein the therapeutic agent is an anti-CTLA-4 antibody.

Embodiment 38 is a method for preventing or reducing cancer metastasis in a patient, the method comprising:

locating at least one lymph node in the patient that intervenes in the lymphatic system between a solid cancer tumor and a draining duct;
placing a medical device comprising a plurality of microneedles on the skin of the patient proximate to a first position under the skin of the patient located between the intervening lymph node and the solid cancer tumor, wherein the first position is proximate to lymph vessels and/or lymph capillaries in the patient's lymphatic system, and wherein the microneedles have a surface comprising nanotopography;
inserting the plurality of microneedles into the patient to a depth whereby at least the epidermis is penetrated and an end of at least one of the microneedles is proximate to the first position; and administering via the plurality of microneedles to the first position a therapeutically effective amount of an anti-CTLA-4 antibody that is effective for preventing or reducing metastasis of the solid cancer tumor.

Embodiment 39 is a method for preventing or reducing cancer metastasis in a patient, the method comprising:

locating a solid cancer tumor in the patient; locating at least one lymph node in the patient that intervenes in the lymphatic system between the solid cancer tumor and a draining duct; placing a medical device that comprises a plurality of microneedles on the skin of the patient at a first location on the skin of the patient that is proximate to lymph capillaries and/or lymph vessels that flow into the intervening lymph node, wherein the microneedles have a surface comprising nanotopography;
inserting the plurality of microneedles into the patient to a depth whereby at least the epidermis is penetrated; and
administering via the plurality of microneedles to the lymph capillaries and/or lymph vessels that flow into the intervening lymph node a therapeutically effective amount of an anti-CTLA-4 antibody that is effective in preventing or reducing cancer metastasis.

Embodiment 40 is a method of treating cancer in a patient, comprising:

placing a medical device comprising a plurality of microneedles on the skin of the patient proximate to a first position under the skin of the patient, wherein the first position is proximate to lymph vessels and/or lymph capillaries in the patient's lymphatic system, and wherein the microneedles have a surface comprising nanotopography;
inserting the plurality of microneedles into the patient to a depth whereby at least the epidermis is penetrated and an end of at least one of the microneedles is proximate to the first position; and administering via the plurality of microneedles to the first position an anti-CTLA-4 antibody, thereby treating the cancer.

Embodiment 41 is the method according to any one of embodiments 38-40, wherein the cancer comprises a tumor.

Embodiment 42 is the method according to any one of embodiments 38-41, wherein the medical device is placed, relative to the tumor, distal to the draining duct.

Embodiment 43 is the method according to embodiment 38-42, wherein at least one lymph node in the patient intervenes in the lymphatic system between the tumor and a draining duct; and the first position is located between the intervening lymph node and the tumor.

Embodiment 44 is the method according to any one of embodiments 38-43, wherein the medical device is placed at a location on the skin of the patient having lymphatic capillaries and/or vessels that flow directly into the intervening lymph node without first passing through any prior lymph node.

Embodiment 45 is the method according to any one of embodiments 38-44, wherein the cancer is a cancer of the head and neck, and the lymph nodes are selected from the group consisting of the jugular lymph nodes, the cervical lymph nodes, the supraclavicular lymph nodes, and combinations thereof.

Embodiment 46 is the method according to any one of embodiments 38-44, wherein the cancer is an oral cavity cancer, and the lymph nodes are selected from the group consisting of the jugular lymph node chain, the cervical lymph nodes, the supraclavicular lymph nodes, and combinations thereof.

Embodiment 47 is the method according to any one of embodiments 38-44, wherein the cancer is a cancer of the pharynx, and the lymph nodes are selected from the group consisting of the jugular lymph node chain, the cervical lymph nodes, the supraclavicular lymph nodes, and combinations thereof.

Embodiment 48 is the method according to any one of embodiments 38-44, wherein the cancer is a melanoma, and the lymph nodes are selected from the group consisting of axillary lymph nodes, inguinal lymph nodes, jugular lymph nodes, cervical lymph nodes, supraclavicular lymph nodes, and combinations thereof.

Embodiment 49 is the method according to any one of embodiments 38-44, wherein the cancer is breast cancer, and lymph nodes are selected from the group consisting of the axillary lymph nodes, the internal mammary lymph nodes, the supraclavicular lymph nodes and combinations thereof.

Embodiment 50 is the method according to any one of embodiments 38-44, wherein the cancer is prostate cancer, and the lymph nodes are selected from the group consisting of the lumbar lymph nodes, the inguinal lymph nodes, the peritoneal lymph nodes and combinations thereof.

Embodiment 51 is the method according to any one of embodiments 38-44, wherein the cancer is in the genital system of the patient with the proviso that it is not ovarian cancer, and the lymph nodes are selected from the group consisting of the lumbar lymph nodes, the inguinal lymph nodes, the peritoneal lymph nodes and combinations thereof.

Embodiment 52 is the method according to any one of embodiments 38-51, wherein the anti-CTLA-4 antibody is imilimumab, a biosimilar thereof, or a bioequivalent thereof.

Embodiment 53 is a method for treating an inflammatory medical condition in a patient, the method comprising:

locating at least one inflammatory locus in the patient, wherein the at least one inflammatory locus comprises lymph vessels, lymph capillaries, lymph nodes, lymph organs or any combination thereof;
locating a first position in the lymphatic system of the patient that is upstream of the inflammatory locus;
placing a medical device comprising a plurality of microneedles on the skin of the patient proximate to the first position, wherein the microneedles have a surface comprising nanotopography;
inserting the plurality of microneedles into the patient to a depth whereby at least the epidermis is penetrated; and
administering via the plurality of microneedles to the first position a therapeutically effective amount of an immune-suppressing agent that is effective in treating the inflammatory medical condition.

Embodiment 54 is the method according to embodiment 53, wherein the upstream position in the lymphatic system is a lymph node selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

Embodiment 55 is the method according to any one of embodiment 53 or 54, wherein the at least one inflammatory locus in the patient is a joint or a psoriatic lesion.

Embodiment 56 is the method according to any one of embodiments 53-55, wherein the at least one inflammatory locus in the patient is a at least one joint selected from the group consisting of an ankle joint, a knee joint, a hip joint, a shoulder joint, an elbow joint, a metacarpophalangeal joint of the hands, a metatarsophalangeal joint in a foot, a wrist joint, a joint in the neck, and combinations thereof.

Embodiment 57 is the method according to any one of embodiments 53-56, wherein the at least one inflammatory locus in the patient is a psoriatic lesion.

Embodiment 58 is the method according to any one of embodiments 53-57, wherein the inflammatory medical condition is selected from the group consisting of Behcet's disease, sarcoidosis, rheumatoid arthritis (RA), juvenile arthritis, psoriatic arthritis, plaque psoriasis, hidradenitis suppurativa, non-infectious uveitis, ankylosing spondylitis, ulcerative colitis (UC), Crohn's disease, and combinations thereof.

Embodiment 59 is a method for lowering the TNF-α level in a patient, the method comprising:

locating a first position in the lymphatic system of the patient;
placing a medical device comprising a plurality of microneedles on the skin of the patient proximate to the first position, and wherein the microneedles have a surface comprising nanotopography;
inserting the plurality of microneedles into the patient to a depth whereby at least the epidermis is penetrated; and
administering via the plurality of microneedles to the first position a therapeutically effective amount of an immune-suppressing agent that is effective in lowering the TNF-α level in the patient.

Embodiment 60 is the method according to embodiment 59, wherein the first position is at least one lymph node of the patient.

Embodiment 61 is a method for treating an inflammatory medical condition in a patient, the method comprising:

locating at least one inflammatory locus in the patient comprising lymph nodes, lymph capillaries, lymph vessel, lymph organs or any combination thereof;
placing a medical device comprising a plurality of microneedles on the skin of the patient proximate to a first position under the skin of the patient, wherein the first position is situated such that it comprises selected lymph capillaries and/or lymph vessels that deliver lymph directly into the lymphatic system in the inflammatory locus, and wherein the microneedles have a surface comprising nanotopography;
inserting the plurality of microneedles into the patient to a depth whereby at least the epidermis is penetrated; and
administering via the plurality of microneedles to the selected lymph capillaries and/or lymph vessels of the patient a therapeutically effective amount of an immune-suppressing agent that is effective in treating the inflammatory medical condition.

Embodiment 62 is a method for treating an inflammatory medical condition in a patient, the method comprising:

placing a medical device comprising a plurality of microneedles on the skin of the patient proximate to a first position under the skin of the patient, wherein the first position is situated such that it comprises lymph capillaries and/or lymph vessels that deliver lymph directly into the lymphatic system, and wherein the microneedles have a surface comprising nanotopography; inserting the plurality of microneedles into the patient to a depth whereby at least the epidermis is penetrated; and
administering via the plurality of microneedles to the lymph capillaries and/or lymph vessels of the patient an immune-suppressing agent, thereby treating the inflammatory medical condition.

Embodiment 63 is the method according to any one of embodiments 53-62, wherein the first position is situated such that it comprises selected lymph capillaries and/or lymph vessels that deliver lymph directly into the lymphatic system in an inflammatory locus in the patient comprising lymph nodes, lymph capillaries, lymph vessel, lymph organs or any combination thereof.

Embodiment 64 is the method according to any one of embodiments 53-63, wherein, relative to the inflammatory locus, the selected lymph capillaries and/or vessels are located distal to the heart of the patient.

Embodiment 65 is the method according to any one of embodiments 53-64, wherein the at least one inflammatory locus in the patient is a joint.

Embodiment 66 is the method according to any one of embodiments 53-65, wherein the at least one inflammatory locus in the patient is at least one joint selected from the group consisting of an ankle joint, a knee joint, a hip joint, a shoulder joint, an elbow joint, a metacarpophalangeal joint of the hands, a metatarsophalangeal joint in a foot, a wrist joint, a joint in the neck, and combinations thereof.

Embodiment 67 is the method according to any one of embodiments 53-66, wherein the inflammatory locus is a knee, and the selected lymph capillaries and/or vessels flow into the popliteal lymph nodes.

Embodiment 68 is the method according to any one of embodiments 53-66, wherein the inflammatory locus is a knee, and relative to the knee, the selected lymph capillaries and/or vessels are located distal to the heart.

Embodiment 69 is the method according to any one of embodiments 53-66, wherein the inflammatory locus is the neck, and the selected lymph capillaries and/or vessels flow into the cervical lymph nodes.

Embodiment 70 is the method according to any one of embodiments 53-66, wherein the inflammatory locus is the neck, and, relative to the neck, the selected lymph capillaries and/or vessels are located distal to the heart.

Embodiment 71 is the method according to any one of embodiments 53-66, wherein the inflammatory locus is a shoulder, and the selected lymph capillaries and/or vessels flow into the pectoral lymph nodes, the superclavical lymph nodes, the axillary lymph nodes or any combination thereof.

Embodiment 72 is the method according to any one of embodiments 53-66, wherein the inflammatory locus is a shoulder, and, relative to the shoulder, the selected lymph capillaries and/or vessels are located distal to the heart.

Embodiment 73 is the method according to any one of embodiments 53-66, wherein the inflammatory locus is an elbow, and the selected lymph capillaries and/or vessels flow into the epitrochlear lymph nodes and/or brachial lymph nodes.

Embodiment 74 is the method according to any one of embodiments 53-66, wherein the inflammatory locus is an elbow, and relative to the elbow, the selected lymph capillaries and/or vessels are located distal to the heart.

Embodiment 75 is the method according to any one of embodiments 53-66, wherein the inflammatory locus is a hip, and the selected lymph capillaries and/or vessels flow into the inguinal lymph nodes and/or the pelvic lymph nodes.

Embodiment 76 is the method according to any one of embodiments 53-66, wherein the inflammatory locus is a hip, and, relative to the hip, the selected lymph capillaries and/or vessels are located distal to the heart.

Embodiment 77 is the method according to any one of embodiments 53-66, wherein the inflammatory locus is a hip, and, relative to the hip, the selected lymph capillaries and/or vessels are located proximate to the heart.

Embodiment 78 is the method according to any one of embodiments 53-77, wherein the inflammatory medical condition is rheumatoid arthritis.

Embodiment 79 is the method according to any one of embodiments 53-78, wherein the inflammatory locus is a psoriatic lesion.

Embodiment 80 is the method according to embodiment 79, wherein the selected lymph capillaries share common lymph vessels and/or lymph capillaries immediately adjacent to and/or within the psoriatic lesion.

Embodiment 81 is the method according to embodiment 79 or 80, wherein the medical device is placed at a location on the skin of the patient having lymph capillaries and/or vessels that flow directly into the lymph nodes within and/or closest to the psoriatic lesion.

Embodiment 82 is the method according to embodiment 81, wherein, relative to the inflammatory locus, the first medical device administers a first therapeutic agent to selected lymph capillaries and/or vessels distal to the heart, and

the method further comprises administering via a second medical device a second therapeutic agent, which is an immune-suppressing agent, to selected lymph capillaries and/or vessels proximate to the heart.

Embodiment 83 is the method according to embodiment 82, wherein the antibody that inhibits TNF-α or etanercept or a biosimilar or bioequivalent thereof administered to the first position and the second therapeutic agent are the same or different.

Embodiment 84 is the method according to any one of embodiments 53-83, wherein the immune-suppressing agent is a TNF-α inhibitor.

Embodiment 85 is the method according to any one of embodiments 53-83, wherein the immune-suppressing agent is adalimumab, adalimumab-atto, certolizumab pegol, etanercept, etanercept-szzs, golimumab, infliximab, infliximab-dyyb, ustekinumab, rituximab, secukinumab, omalizumab, natalizumab, ixekizumab, obinutuzumab, rituximab/hyaluronidase human, or a biosimilar or bioequivalent of any of the foregoing.

Embodiment 86 is the method according to any one of embodiments 53-83, wherein the immune-suppressing agent is an antibody that inhibits TNF-α or etanercept or a biosimilar or bioequivalent thereof.

Embodiment 87 is the method according to any one of embodiments 53-83, wherein the immune-suppressing agent is adalimumab or a biosimilar or bioequivalent thereof.

Embodiment 88 is the method according to any one of embodiments 53-83, wherein the immune-suppressing agent is etanercept or a biosimilar or bioequivalent thereof.

Embodiment 89 is the method according to embodiment 86, wherein the antibody that inhibits TNF-α or etanercept or a biosimilar or bioequivalent thereof is adalimumab or a biosimilar or bioequivalent thereof.

Embodiment 90 is the method according to embodiment 86, wherein the antibody that inhibits TNF-α or etanercept or a biosimilar or bioequivalent thereof is etanercept or a biosimilar or bioequivalent thereof.

Embodiment 91 is the method according to any one of embodiments 53-90, wherein the first medical device is placed at a location on the skin of the patient having lymph capillaries and/or vessels that flow directly into the lymph nodes within and/or closest to the inflammatory locus.

Embodiment 92 is the method according to any one of embodiments 53-91, wherein the selected lymph capillaries and/or vessels, relative to the inflammatory locus, are distal to the heart.

Embodiment 93 is the method according to any one of embodiments 53-92, wherein the selected lymph capillaries and/or vessels, relative to the inflammatory locus, are proximate to the heart.

Embodiment 94 is the method according to any one of embodiments 1-93, wherein the patient is a mammal.

Embodiment 95 is the method according to any one of embodiments 1-94, wherein the patient is a human.

Embodiment 96 is the method according to any one of embodiments 1-95, wherein the medical device is a Sofusa™ drug delivery platform.

Embodiment 97 is the method according to any one of embodiments 1-96, wherein the medical device comprises a fluid delivery apparatus,

wherein the fluid delivery apparatus comprises:
a fluid distribution assembly wherein a cap assembly is coupled to a cartridge assembly, and the cartridge assembly is slidably coupled to a plenum assembly, and a mechanical controller assembly is slidably coupled to the cartridge assembly;
a collet assembly constituting the housing of the fluid delivery apparatus and being slidably coupled to the fluid distribution assembly; and
a plurality of microneedles fluidically connected with the fluid distribution assembly having a surface comprising nanotopography, the plurality of microneedles being capable of penetrating the stratum corneum of the skin of a patient and controllably delivering the therapeutic agent, the anti-CTLA-4 antibody, or the immune-suppressing agent to a depth below the surface of the skin.

Embodiment 98 is the method according to any one of embodiments 1-97, wherein the medical device delivers the therapeutic agent or the anti-CTLA-4 antibody or the immune-suppressing agent to a depth below the surface of the skin of from about 50 μm to about 4000 μm, from about 250 μm to about 2000 μm, or from about 350 μm to about 1000 μm.

Embodiment 99 is the method according to any one of embodiments 1-98, wherein each of the microneedles in the medical device has a length between about 200 to about 800 μm, between about 250 to about 750 μm, or between about 300 to about 600 μm.

### BRIEF DESCRIPTION OF THE DRAWINGS

FIG. 1 illustrates the location of key lymph nodes in the rat.

FIG. 2 is a graph of the PK profile of etanercept in rat blood serum.

FIGS. 3A, 3B, 3C and 3D are graphs illustrating the biodistribution of etanercept in a rat model. FIG. 3A illustrates the biodistribution of etanercept administered by either intravenous (“IV”) or the Sofusa™ drug delivery platform after 12 hours. FIG. 3B illustrates the biodistribution of etanercept administered by either subcutaneous (“SC”) or the Sofusa™ drug delivery platform after 12 hours. FIG. 3C illustrates the biodistribution of etanercept administered by either IV or the Sofusa™ drug delivery platform after 36 hours. FIG. 3D illustrates the biodistribution of etanercept administered by either SC or the Sofusa™ drug delivery platform after 36 hours.

FIG. 4 is a graph summarizing the biodistribution of etanercept in the axillary and inguinal lymph nodes when administered by intravenous, subcutaneous, intradermal (“ID”) or the Sofusa™ drug delivery platform.

FIG. 5A shows a timeline for induction, measurements, and treatments of animals with collagen-induced arthritis.

FIGS. 5B-5G are graphs showing the lymphatic pumping rate (y-axis) versus % joint swelling (x-axis) from the CIA RA rat model comparing the Sofusa™ drug delivery platform to subcutaneous administration of etanercept measured at day 11 (FIGS. 5B and 5C), day 13 (FIGS. 5D and 5E), and day 18 (FIGS. 5F and 5G) respectively from the CIA injection.

FIG. 6 is a graph of the lymphatic pumping rate after the administration of etanercept by either subcutaneous or the Sofusa™ drug delivery platform.

FIGS. 7A, 7B, 7C and 7D are a series of graphs illustrating the lymphatic pumping rate after the administration of etanercept when administered by the Sofusa™ drug delivery platform (FIG. 7A), subcutaneous injection (FIG. 7B), untreated control (FIG. 7C), and intradermal injection (FIG. 7D).

FIG. 8 is comparison of the PK curves for etanercept when administered either via the Sofusa™ drug delivery platform or subcutaneous injection.

FIG. 9 is a graph of the PK profile of etanercept for intravenous, and subcutaneous administration as compared to the Sofusa™ drug delivery platform over 48 hours.

FIG. 10 is a graph comparing the PK/PD profile of etanercept for intravenous, subcutaneous, and intradermal administration as compared to the Sofusa™ drug delivery platform.

FIG. 11 is a series of bioluminescent images showing the metastatic burden on mice treated with either vehicle or an anti-mCTLA-4 monotherapy administered with the Sofusa™ drug delivery platform.

FIG. 12 is a graph comparing tumor volume in rats treated with anti-mCTLA-4 monotherapy by IP administration or the Sofusa™ drug delivery platform.

FIG. 13 is a sectional view of an exemplary fluid delivery apparatus in a pre-use configuration.

FIG. 14 is a sectional view of the fluid delivery apparatus in a pre-activated configuration.

FIG. 15 is an exploded, sectional view of fluid delivery apparatus.

FIG. 16 is a sectional view of a collet assembly of the fluid delivery apparatus.

FIG. 17 is an exploded, perspective view of the collet assembly shown in FIG. 16.

Unless otherwise indicated, the drawings provided herein are meant to illustrate features of embodiments of the disclosure or results of representative experiments illustrating some aspects of the subject matter disclosed herein. These features and/or results are believed to be applicable in a wide variety of systems comprising one or more embodiments of the disclosure. As such, the drawings are not meant to include all additional features known by those of ordinary skill in the art to be required for the practice of the embodiments, nor are they intended to be limiting as to possible uses of the methods disclosed herein.

### DETAILED DESCRIPTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims. The section headings used herein are for organizational purposes only and are not to be construed as limiting the desired subject matter in any way. In the event that any literature incorporated by reference contradicts any term defined in this specification, this specification controls.

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise. Numeric ranges are inclusive of the numbers defining the range.

As used herein, positional terms such as upward, downward, upper, lower, top, bottom, and the like are used only for convenience to indicate relative positional relationships.

### I. Definitions

The terms “medicament”, “medication”, “medicine”, “therapeutic agent” and “drug” are used interchangeably herein and describe a pharmaceutical composition or product intended for the treatment of a medical condition having at least one symptom. The pharmaceutical composition or product will have a physiological effect on the patient when it is introduced into the body of a patient. The pharmaceutical composition can be in any suitable formulation unless a specific formulation type is required or disclosed. In some instances, the medicament will be approved by the US FDA while in other instances it may be experimental (e.g., clinical trials) or approved for use in a country other than the United States (e.g., approved for use in China or Europe). In instances where these terms are used, it is understood that they refer to both singular and plural instances. In some embodiments herein, two or more medicaments may be used in a form of combination therapy. In all cases, the selection of the proper medicament (singular or plural) will be based on the medical condition of the patient and the assessment of the medical professional administering, supervising and/or directing the treatment of the patient. Combination therapies are sometimes more effective than a single agent and used for many different medical conditions. It is understood that combination therapies are encompassed herein and envisioned with the subject matter disclosed.

An “effective amount” or a “therapeutically effective dose” in reference to a medicament is an amount sufficient to treat, ameliorate, or reduce the intensity of at least one symptom associated with the medical condition. In some aspects of this disclosure, an effective amount of a medicament is an amount sufficient to effect a beneficial or desired clinical result including alleviation or reduction in one or more symptoms of a medical condition. In some embodiments, an effective amount of the medicament is an amount sufficient to alleviate all symptoms of a medical condition. In some aspects, a dose of the therapeutic agent will be administered that is not therapeutically effective by itself. In these aspects, multiple doses may be administered to the patient either sequentially (using the same device or different devices) or simultaneously such that the combination of the individual doses is therapeutically effective. For simultaneous administration, additional medical devices comprising a plurality of microneedles or an entirely different route of administration may be used.

The term “patient” as used herein refers to a warm blooded animal such as a mammal which is the subject of a medical treatment for a medical condition that causes at least one symptom. It is understood that at least humans, dogs, cats, and horses are within the scope of the meaning of the term. Preferably, the patient is human.

As used herein, the terms “distal” and “proximal” are used in their anatomical sense. Distal means a given position or structure is situated farther from the center of the body or point of attachment of the limb when compared to another position or structure. Proximal is the opposite of distal. Proximal means a given position or structure is situated closer to the center of the body or point of attachment of the limb when compared to another position or structure. For example, the wrist is distal to the elbow and the shoulder is proximal to the elbow.

As used herein, the term “treat” or “treatment”, or a derivative thereof, contemplates partial or complete amelioration of at least one symptom associated with the medical condition of the patient. “Preventing” a medical condition from occurring (e.g., cancer metastasis) is considered a form of treatment. “Reducing” the incidence of a medical condition (e.g., cancer metastasis) is considered a form of treatment.

Etanercept is a fusion protein produced by recombinant DNA sold under the trade name of Enbrel®. It fuses the TNF receptor to the constant end of the IgG1 antibody, and, when administered to a patient, reduce the effect of naturally present TNF. As such, it is considered a TNF inhibitor. In the United States, it has been approved for clinical use by the FDA for the treatment of moderate to severe rheumatoid arthritis (RA), moderate to severe polyarticular juvenile rheumatoid arthritis (JRA), psoriatic arthritis, ankylosing spondylitis, and moderate to severe plaque psoriasis. Due to the serious number of secondary infections associated with Enbrel®, the FDA requires a black box warning—the most serious level of warning possible under current FDA guidelines. As used herein, the term etanercept and Enbrel® are used interchangeably and also encompass any biosimilars or bioequivalents thereof.

Checkpoint inhibitors are a form of cancer therapy that directly affect the functioning of the immune system of the patient. Immune system checkpoints can be either stimulatory or inhibitory, and some cancers are known to affect these checkpoints to prevent the immune system from attacking them. As such, checkpoint inhibitors can block these inhibitory checkpoints thereby restoring proper immune system function. Examples of checkpoints include, but are not limited to, CTLA-4, PD-1, and PD-L1. Some checkpoint inhibitors that are currently approved by the FDA include, but are not limited to, ipilimumab (CTLA-4 inhibitor; sold under the tradename of Yervoy®), nivolumab (PD-1 inhibitor; sold under the tradename of Opdivo), pembrolizumab (PD-1 inhibitor; sold under the tradename of Keytruda®), and atezolizumab (PD-L1 inhibitor; sold under the tradename of Tecentriq®). As used herein, the term checkpoint inhibitor encompasses medicaments that are used to inhibit an immune system checkpoint and restore immune system function.

As used herein, “bioavailability”, means the total amount of a given dosage of the administered agent that reaches the blood compartment. This is generally measured as the area under the curve (AUC) in a plot of concentration vs. time.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art, see, e.g., the Physicians' Desk Reference (69th ed., 2015), which is incorporated herein by reference in its entirety.

Cₘₐₓ refers to the maximum concentration that a medicament achieves in the plasma or tissue of a patient after the medicament has been administered while Ct refers to the concentration that a medicament achieves at a specific time (t) following administration. Unless otherwise stated, all discussion herein is in regard to pharmacokinetic parameters in plasma.

The AUCₜ refers to the area under the plasma concentration time curve from time zero to time t following administration of the medicament.

The AUC_{∞} refers to the area under the plasma concentration time curve from time zero to infinity (infinity meaning that the plasma concentration of the medicament is below detectable levels).

Tₘₐₓ is the time required for the concentration of a medicament to reach its maximum blood plasma concentration in a patient following administration. Some forms of administration of a medicament will reach their Tₘₐₓ slowly (e.g., tablets and capsules taken orally) while other forms of administration will reach their Tₘₐₓ almost immediately (e.g., subcutaneous and intravenous administration).

“Steady state” refers to the situation where the overall intake of a drug is approximately in dynamic equilibrium with its elimination.

A discussion of various pharmacokinetic parameters and the methods of measuring and calculating them can be found in Clinical Pharmacokinetics and Pharmacodynamics: Concepts and Applications, M. Rowland and T. N. Tozer, (Lippincott, Williams & Wilkins, 2010) which is incorporated by reference for its teachings thereof.

“Or” is used in the inclusive sense, i.e., equivalent to “and/or,” unless the context requires otherwise.

### II. Lymphatic System Delivery Methods

In the methods disclosed herein, two different exemplary modes for delivering a therapeutic agent to a patient are envisioned. In one mode, the target for the therapeutic agent is clearly identified, and the medical device comprising a plurality of microneedles is placed such that the medicament is administered to the lymphatic system of the patient such that it is carried by the lymph vessels directly to that target. The target may be, e.g., a solid tumor or a specifically inflamed joint in a patient. In this case, while some systemic exposure will occur, the administration is much more regionalized. In the second mode, the therapeutic target or exact location of the target may be unknown or less clearly defined, delivery of the therapeutic agent is into the lymphatic system of the patient, and the agent is intended to traverse the lymphatic system to either the right lymphatic duct or the thoracic duct. The therapeutic agent then enters the circulatory system of the patient leading to systemic exposure to the agent. For example, if a solid tumor has metastasized, the location of secondary sites for these cancer cells may not be known. Also, for some inflammatory medical conditions (e.g., Crohn's disease), an exact target for delivery of the therapeutic agent is not known. Although the therapeutic agent may traverse certain lymph nodes before reaching either of the draining ducts, the administration is considered to result in systemic exposure. As such, one skilled in the art can apply methods disclosed to provide targeted, regional administration of a therapeutic agent or more widespread systemic administration. A medical professional can determine which mode of administration is appropriate for an individual patient and place the medical device or devices accordingly.

In some aspects, the therapeutic target is a lymph node, a lymph vessel, an organ that is part of the lymphatic system or a combination thereof. In some aspects, the therapeutic target is a lymph node. In some aspects, the therapeutic target is a specific lymph node as described elsewhere herein.

In some embodiments, delivery of the therapeutic agent to the lymphatic system is delivery into the vessels of the lymphatic vasculature, the lymph nodes as described elsewhere herein, or both. In some aspects, delivery is to the superficial lymph vessels. In yet another aspect, delivery is to one or more lymph nodes. The specific target for delivery will be based on the medical needs of the patient.

In patients where more than one medical device is used to deliver the therapeutic agent to a plurality of locations on the body of a patient, the overall dose of the therapeutic agent at each location must be carefully adjusted such that the patient does not receive an overall unsafe combined dose of the agent. Being able to more selectively target specific locations in or on the body of a patient more precisely often means a lower dose is required at each specific location. In some embodiments, the dose administered to target one or more locations on the body of a patient is lower than a dose administered by other routes, including intravenous and subcutaneous administration.

Because the lymph fluid circulates throughout the body of a patient in a similar manner to blood in the circulatory system, any single position in the lymphatic vasculature can be upstream or downstream relative to another position. As used herein in reference to the lymphatic vasculature, the term “downstream” refers to a position in the lymphatic system closer (as the fluid travels through the vessels in a healthy patient) to either the right lymphatic duct or the thoracic duct relative to the reference position (e.g., a tumor or internal organ or a joint). As used herein, the term “upstream” refers to a position in the lymphatic system that is farther from the right lymphatic duct or the thoracic duct relative to the reference position. Because the direction of fluid flow in the lymphatic system can be impaired or reversed due to the medical condition of the patient, the terms “upstream” and “downstream” do not specifically refer to the direction of fluid flow in the patient undergoing medical treatment. They are positional terms based on their physical position relative to the draining ducts as described.

Because lymph nodes often occur in a group as opposed to being present as a single isolated node, the term “lymph node” as used herein can be singular or plural and refer to either a single isolated lymph node or a group of lymph nodes in a small physical location. For example, a reference to the inguinal lymph node or inguinal lymph nodes refers to the group of lymph nodes that are recognized by a person skilled in the art (i.e., a medical professional such as a doctor or a nurse) as a group of lymph nodes located in the hip/groin area or femoral triangle in a patient. It also refers to both the superficial and deep lymph nodes unless specifically stated otherwise. In some aspects, the lymph node is the sentinel lymph node for a specific solid cancer tumor.

In some embodiments, the lymph node is selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, the medicament is delivered to the interstitium of the patient, e.g., to a space between the skin and one or more internal structures, such as an organ, muscle, or vessel (artery, vein, or lymph vessel), or any other spaces within or between tissues or parts of an organ. In still yet another embodiment, the medicament is delivered to both the interstitium and the lymphatic system. In embodiments where the therapeutic agent is delivered to the interstitium of the patient, it may not be necessary to locate the lymph nodes or lymphatic vasculature of the patient before administering the therapeutic agent.

### III. Administration of a Therapeutic Agent to Multiple Regions of the Lymphatic System

One embodiment disclosed herein is a method for administering a therapeutic agent to the lymphatic system of a patient. The method generally comprises placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography; placing a second medical device comprising a plurality of microneedles on the skin of the patient at a second location proximate to a second position under the skin of the patient, wherein the second position is proximate to lymph vessels and/or lymph capillaries that drain into the thoracic duct, and wherein the microneedles of the second medical device have a surface comprising nanotopography; inserting the plurality of microneedles of the first medical device into the patient to a depth whereby at least the epidermis is penetrated and an end of at least one of the microneedles is proximate to the first position; inserting the plurality of microneedles of the second medical device into the patient to a depth whereby at least the epidermis is penetrated and an end of at least one of the microneedles is proximate to the second position; and administering via the microneedles of the first medical device a first dose of the therapeutic agent into the first position; administering via the microneedles of the second medical device a second dose of the therapeutic agent into a second position; wherein administering the doses cumulatively provides a therapeutically effective amount of the therapeutic agent.

In another aspect, disclosed herein is a method for administering a therapeutic agent to the lymphatic system of a patient. The method generally comprises placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography; placing a second medical device comprising a plurality of microneedles on the skin of the patient at a second location proximate to a second position under the skin of the patient, wherein the second position is proximate to lymph vessels and/or lymph capillaries that drain into the thoracic duct, and wherein the microneedles of the second medical device have a surface comprising nanotopography; inserting the plurality of microneedles of the first medical device into the patient to a depth whereby at least the epidermis is penetrated and an end of at least one of the microneedles is proximate to the first position; inserting the plurality of microneedles of the second medical device into the patient to a depth whereby at least the epidermis is penetrated and an end of at least one of the microneedles is proximate to the second position; administering via the microneedles of the first medical device a first therapeutically effective dose of the therapeutic agent into the first position; and administering via the microneedles of the second medical device a second therapeutically effective dose of the therapeutic agent into the second position; wherein a beginning time for administering the first dose and the second dose are different and separated by a period of time.

In some aspects disclosed herein, the first position and second position are reversed and the first position is proximate to lymph vessels and/or lymph capillaries that drain into the thoracic duct and the second position is proximate to lymph vessels and/or lymph capillaries that drain into the right lymphatic duct. As noted, one medical device drains into one of the two draining ducts in the lymphatic system while the other medical device drains into the other draining duct. This method is envisioned to administer at least a therapeutic agent to the lymphatic system of the patient such that different parts of the lymphatic system are exposed to the therapeutic agent. In some aspects, two or more medical devices are placed such that they drain into the same draining duct but they target different regions of the lymphatic system of the patient. For example, one device may be placed on the left arm of the patient and one device may be placed on the left leg of the patient. Although the therapeutic agent would ultimately drain through the same duct for site of administration, the therapeutic agent would traverse significantly different regions of the lymphatic system of the patient.

In some aspects, the first dose of the therapeutic agent and the second dose of the therapeutic agent are not therapeutically effective individually, but the combined amount of the doses is therapeutically effective. The first dose and the second dose can be administered sequentially or simultaneously. In some aspects, the first dose and the second dose are administered sequentially. In some aspects, the first dose and the second dose are administered simultaneously. In some aspects, administration of the two doses at least partially overlaps in time. This means that the administration of the two doses commences at different times, but the administration of the second dose begins before the administration of the first dose ends.

The location on the body of the patient is selected based on the medical condition of the patient and the knowledge of the medical professional supervising, directing and/or administering the treatment. For each medical device used with the methods disclosed herein, the location of the medical device on the body of the patient is selected independently of the other medical devices with the caveat that the objective of this method is to expose different parts of the lymphatic system to the therapeutic agent. In some aspects, each medical device is placed on a limb (i.e., arm or leg) of the patient. In order to achieve maximum exposure of the lymphatic system to the therapeutic agent, one device is placed on the right arm of the patient while the other device is place on the left leg of the patient. Alternatively, one device could be placed on the left arm of the patient while the other device is placed on the right leg of the patient. In yet another aspect, one medical device is placed on the right arm of the patient while the other medical device is placed on either the left arm or left leg of the patient. In yet another aspect, one medical device is placed on the left arm of the patient and the other medical device is placed on the right arm or right leg of the patient. A device on the arm of the patient may be located proximate to the wrist or hand of the patient while a device on the patient may be located proximate to the ankle or foot of the patient.

In still yet another aspect, the methods disclosed herein further comprise placing a third medical device comprising a plurality of microneedles on the skin of the patient at a third location proximate to a third position under the skin of the patient, wherein the third position is proximate to lymph vessels and/or lymph capillaries; inserting the plurality of microneedles of the third medical device into the patient to a depth whereby at least the epidermis is penetrated and an end of at least one of the microneedles is proximate to the third position; and administering via the third medical device a third dose of said therapeutic agent; and wherein the third location is different than the first location and the second location, and the third position is different that the first position and the second position.

In still yet another aspect, the methods disclosed herein further comprise placing a fourth medical device comprising a plurality of microneedles on the skin of the patient at a fourth location proximate to a fourth position under the skin of the patient, wherein the fourth position is proximate to lymph vessels and/or lymph capillaries; inserting the plurality of microneedles of the fourth medical device into the patient to a depth whereby at least the epidermis is penetrated and an end of at least one of the microneedles is proximate to the fourth position; and administering via the fourth medical device a fourth dose of said therapeutic agent; and wherein the first location, the second location, the third location, and the fourth location are on different limbs of the patient.

For any of the methods disclosed in, including those that use two medical devices, three medical devices, or four medical devices, in some aspects, each medical device is placed such that it initially drains into different lymph nodes, and wherein the draining lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes.

In one non-limiting example where three medical devices are used on a patient, the first device is placed on the right forearm of the patient which would then drain into the right axillary lymph nodes; the second device is placed on the left forearm of the patient which would then drain into the left axillary lymph nodes; and the third device is placed on the left thigh of the patient which would then drain into the left inguinal lymph nodes. In this instance the second and third devices would both drain into the thoracic duct but the initial draining lymph nodes are different.

In some aspects, the first dose of the therapeutic agent, the second dose of the therapeutic agent, and if present, the third dose of the therapeutic agent and the fourth dose of the therapeutic agent may each be administered to the patient sequentially or simultaneously. Doses may be combined such that the first and second dose are administered simultaneously while the third and fourth dose are administered together but sequentially relative to the first and second doses. In another aspect, the first and third dose and simultaneously administered while the second and fourth dose are administered simultaneous with each other and sequentially with the first and third dose. In yet another aspect, each dose is administered sequentially.

For any individual dose or combination of doses that are administered sequentially, there is a predetermined period of time between the beginning of each administration. That predetermined period of time may be 15 minutes, 30 minutes, 45 minutes, 1 hour, 2 hours, 3 hours, 4 hours, 5 hours, 6 hours, 7 hours, 8 hours, 12 hours, 16 hours, 20 hours, 24 hours, 36 hours, 48 hours, 60 hours, or 72 hours. The predetermined period may be from about 15 minutes to about 72 hours or a time increment therebetween. Each period of time is selected independently of any other period of time and is based on the medical needs of the patient and the assessment of the medical professional administering, supervising or directing the treatment of the patient. Because the time that it takes to administer a dose of the therapeutic agent with the medical device is not zero, it is possible that the initiation of administering a subsequent dose of the therapeutic agent will be before the completion of the administration of the prior dose. For example, the administration of the second dose of the therapeutic agent may begin before the administration of the first dose of the therapeutic agent is complete. In yet another aspect, the predetermined period of time is based on the ending of one dose and the initiation of the next dose.

In some embodiments, the anti-CTLA-4 antibody is imilimumab, a biosimilar thereof, or a bioequivalent thereof. If two or more medical devices are used, the anti-CTLA-4 antibody administered to the patient using the two or more devices may be the same or different.

In some aspects, the therapeutic agent is effective in treating or relieving the symptoms of an inflammatory medical condition. In some aspects, the therapeutic agent is an antibody that inhibits TNF-α. In some embodiments, the therapeutic agent is adalimumab, adalimumab-atto, certolizumab pegol, etanercept, etanercept-szzs, golimumab, infliximab, infliximab-dyyb, or a biosimilar or bioequivalent of any one of the foregoing agents. In some embodiments, the therapeutic agent is etanercept, a biosimilar thereof, or a bioequivalent thereof. In some embodiments, the therapeutic agent is adalimumab, a biosimilar thereof, or a bioequivalent thereof. In some embodiments, the therapeutic agent is an immune-suppressing agent. In some embodiments, the immune-suppressing agent is adalimumab (Humira®), etanercept (Enbrel®), infliximab (Remicade®), ustekinumab (Stelara®), rituximab (Rituxan®), secukinumab) (Cosentyx®, omalizumab (Xolair®), natalizumab (Tysabri®), ixekizumab (Taltz®), obinutuzumab (Gazyve®), or rituximab/hyaluronidase human (Rituxan Hycela™), or a biosimilar or bioequivalent of any of the foregoing.

In still yet another embodiment, disclosed herein is a method for increasing the bioavailability of a therapeutic agent in a patient, the method comprising placing at least one medical device that comprises a plurality of microneedles on the skin surface of the patient; and administering a therapeutic agent with the at least one medical device to the patient.

In some embodiments, the methods for delivering a therapeutic agent to a patient as described herein result in an equivalent blood serum absorption rate of one or more therapeutic agents described herein as compared to intravenous, subcutaneous, intramuscular, intradermal or parenteral delivery routes while retaining relatively higher rates of lymphatic delivery as described herein. Without being bound by any theory, the rate of delivery and increased bioavailability may be due to the lymphatic circulation of one or more agents through the thoracic duct or the right lymphatic duct and into the blood circulation. Standard highly accurate and precise methodologies for measuring blood serum concentration and therapeutic monitoring at desired time points may be used that are well known in the art, such as radioimmunoassays, high-performance liquid chromatography (HPLC), fluorescence polarization immunoassay (FPIA), enzyme immunoassay (EMIT) or enzyme-linked immunosorbant assays (ELISA). For calculating the absorption rate using the methods described above, the drug concentration at several time points should be measured starting immediately following administration and incrementally thereafter until a Cₘₐₓ value is established and the associated absorption rate calculated.

### IV. Methods for Treating Cancer and/or Preventing Cancer Metastasis by Lymphatic Delivery

In some embodiments, a method for treating cancer metastasis in a patient is provided. The method may comprise locating at least one lymph node in the patient that intervenes in the lymph system between a solid cancer tumor and a draining duct; placing a medical device comprising a plurality of microneedles on the skin of the patient proximate to a first position under the skin of the patient located between the intervening lymph node and the solid cancer tumor, wherein the first position is proximate to lymph vessels and/or lymph capillaries in the patient's lymphatic system, and wherein the microneedles have a surface comprising nanotopography; inserting the plurality of microneedles into the patient to a depth whereby at least the epidermis is penetrated and an end of at least one of the microneedles is proximate to the first position; and administering via the plurality of microneedles to the first position a therapeutically effective amount of an anti-CTLA-4 antibody.

In yet another embodiment, disclosed herein is a method for treating, preventing or reducing cancer metastasis in a patient. The method may comprise locating a solid cancer tumor in the patient; locating at least one lymph node in the patient that intervenes in the lymph system between the solid cancer tumor and a draining duct; placing a medical device that comprises a plurality of microneedles on the skin of the patient at a first location on the skin of the patient that is proximate to lymph capillaries and/or lymph vessels that flow into the intervening lymph node, and wherein the microneedles have a surface comprising nanotopography; inserting the plurality of microneedles into the patient to a depth whereby at least the epidermis is penetrated; and administering via the plurality of microneedles to the lymph capillaries and/or lymph vessels that flow into the intervening lymph node a therapeutically effective amount of an anti-CTLA-4 antibody.

In some embodiments, a method of treating cancer in a patient is provided. The method comprises placing a medical device comprising a plurality of microneedles on the skin of the patient proximate to a first position under the skin of the patient, wherein the first position is proximate to lymph vessels and/or lymph capillaries in the patient's lymphatic system, and wherein the microneedles have a surface comprising nanotopography; inserting the plurality of microneedles into the patient to a depth whereby at least the epidermis is penetrated and an end of at least one of the microneedles is proximate to the first position; and administering via the plurality of microneedles to the first position an anti-CTLA-4 antibody, thereby treating the cancer.

In some embodiments, a method disclosed herein comprising administering an anti-CTLA-4 antibody has any of the features set forth above with respect to methods of administering a therapeutic agent to the lymphatic system of a patient, e.g., including administration into first and second positions that are proximate to lymph vessels and/or lymph capillaries that drain into the right lymphatic duct and the thoracic duct, respectively.

In some aspects, the lymph node is the cancer draining lymph node. Cancer draining lymph node refers to a lymph node that is downstream from a solid cancer tumor and is the first lymph node impacted by metastasis of the tumor. The first lymph node affected by metastasis is often referred to as the sentinel lymph node.

Because metastasis can be a systemic issue for a patient rather than just a localized one, in some aspects the medical device is placed on the patient to effect systemic delivery of the anti-CTLA-4 antibody rather than just targeted delivery to an identified lymph node. In some aspects, the device is placed such that the anti-CTLA-4 antibody is not targeting a specific lymph node, although it may traverse one or more lymph nodes after administration; the medical device is placed with the expectation that the anti-CTLA-4 antibody will enter the circulatory system of the patient after traversing the lymphatic vasculature leading to systemic exposure to the anti-CTLA-4 antibody. This type of administration is intended to treat metastasized cancer cells that have moved past the local environment of the primary solid cancer tumor. Such metastasized cancer cells may not yet be exhibiting symptoms in that new location, but eventually will if left untreated.

In yet another aspect, disclosed herein is a method for treating a solid cancer tumor in a patient. The method generally comprises locating the solid cancer tumor in the patient; locating a position in the lymphatic system of the patient that is upstream of the solid cancer tumor; placing a medical device comprising a plurality of microneedles on the skin of the patient proximate to a first position under the skin of the patient located proximate to the position in the lymphatic system of the patient that is upstream of the solid cancer tumor, wherein the first position is proximate to lymph vessels and/or lymph capillaries that are upstream of the solid cancer tumor, and wherein the microneedles have a surface comprising nanotopography; inserting the plurality of microneedles into the patient to a depth whereby at least the epidermis is penetrated; and administering via the plurality of microneedles to the first position a therapeutically effective amount of an anti-CTLA-4 antibody that is effective for preventing or reducing metastasis of the solid cancer tumor. In some aspects, the position in the lymphatic system of the patient to which the therapeutic agent is delivered is downstream of the solid cancer tumor.

In some embodiments, administering is done to the lymph vessels upstream to the solid cancer tumor. In other embodiments, administering is done to both the lymph nodes and lymph vessels upstream of the solid cancer tumor. In some aspects, it may not be necessary to locate a lymph node upstream of the tumor before administering the anti-CTLA-4 antibody to the patient. In some embodiments, the medical device is placed distal to the draining duct relative to the solid cancer tumor. In yet another aspect, the medical device is proximal to the draining duct relative to the solid cancer tumor.

Because cancer and other medical conditions can damage the lymphatic system of a patient, the flow of fluid in the lymphatic system can be impaired or even reversed (called backflow). This can lead to swelling in the surrounding tissues and organs of the patient. In some aspects, the medical device is placed such that backflow in the lymphatic system transports the anti-CTLA-4 antibody to the targeted location. For example, in a properly functioning lymphatic system, the downstream position relative to a solid cancer tumor would not transport the anti-CTLA-4 antibody directly into the tumor. However, in an impaired lymphatic system, backflow from a downstream position relative to the solid cancer tumor would transport the anti-CTLA-4 antibody directly to the tumor. A medical professional skilled in the art understands the manner by which the lymphatic system functions and will make treatment decisions for the patient based on that knowledge.

In some aspects, the medical device is placed at a location on the skin of the patient such that the lymph vessels and/or capillaries flow directly into a specifically targeted lymph node without first passing through the solid cancer tumor or any other lymph nodes. In this instance, the anti-CTLA-4 antibody, after administration, would enter the lymph vessels of the patient and flow directly into the targeted lymph node. In yet another aspect, there may be lymph nodes between the site of administration and the targeted lymph nodes. One nonlimiting example when this occurs is if the targeted lymph node is deep within the body of the patient and there are no lymph vessels near the skin of the patient that flow directly into the targeted lymph node.

It is known that certain types of cancer often metastasize to specific lymph nodes, and the placement of the medical device may be made on this basis. For example, oral and pharynx cancers, in addition to those of the head and neck, metastasize to the jugular lymph node chain, the cervical lymph nodes and the supraclavicular lymph nodes; many skin cancers (e.g., melanomas) metastasize to the draining axillary and/or inguinal lymph node basins depending on the location of the cancer; breast cancer metastasizes to the axillary, internal mammary and supraclavicular lymph nodes; prostate cancer metastasizes to the lumbar, inguinal and peritoneal lymph nodes; brain and central nervous system cancers metastasize into the jugular, cervical and lumbar lymph nodes; ovarian cancers metastasize to the retroperitoneal (pelvic and/or para-aortic) lymph nodes; cancer in the genitals of a patient metastasize to the lumbar lymph nodes, the inguinal lymph nodes, and the peritoneal lymph nodes.

The specific lymph node targeted for delivery of the medicament is based on any reasonable criteria based on the medical needs and condition of the patient. For example, a lymph node biopsy may be performed to determine if metastatic cancer cells are present in a specific lymph node. Alternatively, a lymph node may be selected based on its location relative to a previously located tumor in the body of a patient. In some embodiments, the lymph node is selected because it is downstream from the solid cancer tumor. Placing the medical device in a position to target the downstream lymph nodes would affect metastatic cancer cells that are in those lymph nodes and reduce the likelihood of their spreading to other parts of the body. Alternatively, the medical device may be placed upstream of the tumor in order to take advantage of tumor-induced lymphangiogenesis that often occurs with solid cancer tumors. In this arrangement, the medicament would flow directly into the tumor thereby more effectively targeting the tumor.

In some aspects, the amount of medicament required to target the metastatic cancer cells or the tumor is lower than the amount given by other routes of administration. A lower dose that is still therapeutically effective may reduce or eliminate side effects leading to a more positive patient outcome.

In some embodiments, the anti-CTLA-4 antibody is imilimumab, biosimilars thereof, bioequivalents thereof. If two or more medical devices are used, the anti-CTLA-4 antibody administered to the patient using the two or more devices may be the same or different. In yet another aspect, two medical devices comprising a plurality of microneedles are used to administer a single anti-CTLA-4 antibody. In this case each individual dose administered by each medical device may be smaller than a therapeutically effective dose, but the combined dose administered by the two medical devices is therapeutically effective.

When the methods disclosed herein are used to treat solid cancer tumors or treat, reduce or eliminate cancer metastasis, the cancer may be any type susceptible to treatment with an anti-CTLA-4 antibody. The type of cancer includes, but is not limited to, adenoid cystic carcinoma, adrenal gland tumor, amyloidosis, anal cancer, ataxia-telangiectasia, atypical mole syndrome, beckwith wiedemann syndrome, bile duct cancer, birt hogg dube syndrome, bladder cancer, bone cancer, brain tumor, breast cancer, carcinoid tumor, carney complex, cervical cancer, colorectal cancer, ductal carcinoma, endometrial cancer, esophageal cancer, familial-adenomatous polyposis, gastric cancer, gastrontestinal stromal tumor, islet cell tumor, juvenile polyposis syndrome, Kaposi's sarcoma, kidney cancer, laryngeal cancer, liver cancer, lobular carcinoma, lung cancer, small cell lung cancer, Hodgkin's lymphoma, non-Hodgkin's lymphoma, lynch syndrome, malignant glioma, mastocytosis, melanoma, meningioma, multiple endocrine neoplasia type 1, multiple Endocrine Neoplasia type 2, multiple myeloma, myelodysplastic syndrome, Nasopharyngeal cancer, Neuroendocrine tumor, Nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma, polycythemia vera, prostate cancer, renal cell cancer, retinoblastoma, salivary gland cancer, sarcoma, alveolar soft part and cardiac sarcoma, Kaposi sarcoma, skin cancer, small bowel cancer, small Intestine cancer, stomach cancer, testicular cancer, thymoma, thyroid cancer, turcot syndrome, uterine (endometrial) cancer, vaginal cancer, von-Hippel-Lindau syndrome, Wilms' tumor (childhood), xeroderma pigmentosum and combinations thereof. In some aspects, the cancer is selected from the group consisting of bladder cancer, breast cancer, cervical cancer, colorectal cancer, esophageal cancer, Hodgkin's lymphoma, non-Hodgkin's lymphoma, kidney cancer, lung cancer, small cell lung cancer, melanoma, oral cancer, pancreatic cancer, pancreatic neuroendocrine tumors, penile cancer, prostate cancer, renal cell cancer, stomach cancer, testicular cancer, thyroid cancer, uterine (endometrial) cancer, and vaginal cancer.

In some aspects, administering the anti-CTLA-4 antibody is directly to a lymph node, a lymph vessel, an organ that is part of the lymphatic system or a combination thereof. In some aspects, administering is to a lymph node. In some aspects, administering is to a specific lymph node as described elsewhere herein. In yet another aspect, administering is to lymph vessels that are upstream of and known to flow into specific lymph nodes. In yet another aspect, administering is to lymph vessels that are upstream of and known to flow into a solid cancer tumor.

It is understood that when multiple doses of an anti-CTLA-4 antibody are administered to a patient, each individual dose may not be therapeutically effective, but the combined doses are therapeutically effective. The combined doses that are therapeutically effective may be smaller than a therapeutically effective dose if the anti-CTLA-4 antibody is administered by a different route (e.g., subcutaneous, intravenous, etc.).

In some embodiments, delivery of the anti-CTLA-4 antibody to the lymphatic system is delivery into the lymphatic vasculature, the lymph nodes as described elsewhere herein, or both. In some aspects, delivery is to the superficial lymph vessels. In yet another aspect, delivery is to one or more lymph nodes. The specific target for delivery will be based on the medical needs of the patient. In one nonlimiting example, if a lymph node biopsy or other medical assessment (e.g., lymph node swelling) is found to be positive for possible metastatic cancer cells, then the medical device comprising a plurality of microneedles can be placed on the patient such that it delivers the anti-CTLA-4 antibody directly to the lymph node. In another nonlimiting example, a sentinel lymph node biopsy is performed where the sentinel lymph nodes are selected based on the type of cancer and the assessment of a medical professional. Alternatively, the medical device can be placed upstream of the lymph node such that the anti-CTLA-4 antibody is delivered to the lymph vessels that feed into the targeted lymph node. In some embodiments, two or more medical devices are used to target two or more different locations in the lymphatic system of the patient. In another nonlimiting example, the medical device is placed upstream of a solid cancer tumor such that the anti-CTLA-4 antibody feeds directly into the tumor. In another example, the medical device is placed directly downstream from a solid cancer tumor such that the anti-CTLA-4 antibody would traverse the same lymphatic vessels as metastatic cells. In yet another aspect, one medical device is placed upstream of the solid cancer tumor and a second medical device is placed downstream of the solid cancer tumor. This would effectively treat both the solid cancer tumor and any possible metastatic cells that have begun to spread in the patient.

### V. Methods for Treating an Inflammatory Medical Condition by Lymphatic Delivery

One embodiment disclosed herein is a method for treating an inflammatory medical condition in a patient. The method generally comprises locating at least one inflammatory locus in the patient, wherein the at least one inflammatory locus comprises lymph vessels, lymph capillaries, lymph nodes, lymph organs or any combination thereof; locating a first position in the lymphatic system of the patient that is upstream of the inflammatory locus; placing a medical device comprising a plurality of microneedles on the skin of the patient proximate to the first position, and wherein the microneedles have a surface comprising nanotopography; inserting the plurality of microneedles into the patient to a depth whereby at least the epidermis is penetrated; and administering via the plurality of microneedles to the first position a therapeutically effective amount of an antibody that inhibits TNF-α or etanercept or a biosimilar or bioequivalent thereof.

In another aspect, disclosed herein is a method for lowering the TNF-α level in a patient. The method generally comprises locating a first position in the lymphatic system of the patient; placing a medical device comprising a plurality of microneedles on the skin of the patient proximate to the first position, and wherein the microneedles have a surface comprising nanotopography; inserting the plurality of microneedles into the patient to a depth whereby at least the epidermis is penetrated; and administering via the plurality of microneedles to the first position a therapeutically effective amount of an antibody that inhibits TNF-α or etanercept or a biosimilar or bioequivalent thereof.

In another aspect, disclosed herein is a method for treating an inflammatory medical condition in a patient. The method generally comprises locating at least one inflammatory locus in the patient comprising lymph nodes, lymph capillaries, lymph vessel, lymph organs or any combination thereof; placing a medical device comprising a plurality of microneedles on the skin of the patient proximate to a first position under the skin of the patient, wherein the first position is situated such that it comprises selected lymph capillaries and/or lymph vessels that deliver lymph directly into the lymphatic system in the inflammatory locus, and wherein the microneedles have a surface comprising nanotopography; inserting the plurality of microneedles into the patient to a depth whereby at least the epidermis is penetrated; and administering via the plurality of microneedles to the selected lymph capillaries and/or lymph vessels of the patient a therapeutically effective amount of an antibody that inhibits TNF-α or etanercept or a biosimilar or bioequivalent thereof.

In some embodiments, a method for treating an inflammatory medical condition in a patient is provided. The method comprises placing a medical device comprising a plurality of microneedles on the skin of the patient proximate to a first position under the skin of the patient, wherein the first position is situated such that it comprises lymph capillaries and/or lymph vessels that deliver lymph directly into the lymphatic system, and wherein the microneedles have a surface comprising nanotopography; inserting the plurality of microneedles into the patient to a depth whereby at least the epidermis is penetrated; and administering via the plurality of microneedles to the lymph capillaries and/or lymph vessels of the patient an antibody that inhibits TNF-α or etanercept or a biosimilar or bioequivalent thereof, thereby treating the inflammatory medical condition.

In some embodiments, a method disclosed herein comprising administering an antibody that inhibits TNF-α or etanercept or a biosimilar or bioequivalent thereof has any of the features set forth above with respect to methods of administering a therapeutic agent to the lymphatic system of a patient, e.g., including administration into first and second positions that are proximate to lymph vessels and/or lymph capillaries that drain into the right lymphatic duct and the thoracic duct, respectively.

In some embodiments, the inflammatory medical condition is selected from the group consisting of Behcet's disease, sarcoidosis, rheumatoid arthritis (RA), juvenile arthritis, psoriatic arthritis, plaque psoriasis, hidradenitis suppurativa, non-infectious uveitis, ankylosing spondylitis, ulcerative colitis (UC), Crohn's disease, and combinations thereof. In some embodiments, the inflammatory medical condition is rheumatoid arthritis. In some embodiments, the inflammatory medical condition is psoriatic arthritis. In some embodiments, the inflammatory medical condition is plaque psoriasis. In some embodiments, the inflammatory medical condition is ulcerative colitis. In some embodiments, the inflammatory medical condition is Crohn's disease. The inflammatory medical condition may be acute or chronic.

The inflammatory locus in the patient can be any location in the patient that exhibits signs of inflammation; such signs include, but are not limited to, redness, swelling, fluid retention, joint pain, joint stiffness, unusual warmth at the location, and loss of joint function.

In some embodiments, administering is done to the lymph vessels upstream to inflammatory locus. In other embodiments, administering is done to both the lymph nodes and lymph vessels upstream of the inflammatory locus. In some aspects, it may not be necessary to locate a lymph node upstream of the inflammatory locus before administering the antibody that inhibits TNF-α or etanercept or a biosimilar or bioequivalent thereof to the patient.

Because some medical conditions can damage the lymphatic system of a patient, the flow of fluid in the lymphatic system can be impaired or even reversed (called backflow). This can lead to swelling in the surrounding tissues and organs of the patient. In some aspects, the medical device is placed such that backflow in the lymphatic system transports the antibody that inhibits TNF-α or etanercept or a biosimilar or bioequivalent thereof to the targeted location. For example, in a properly functioning lymphatic system, the downstream position relative to an inflammatory locus would not transport the antibody that inhibits TNF-α or etanercept or a biosimilar or bioequivalent thereof directly into the inflammatory locus. However, in an impaired lymphatic system, backflow from a downstream position relative to the inflammatory locus would transport the antibody that inhibits TNF-α or etanercept or a biosimilar or bioequivalent thereof directly to the inflammatory locus. A medical professional skilled in the art understands the manner by which the lymphatic system functions and will make treatment decisions for the patient based on that knowledge.

In some aspects, the inflammatory locus is a joint, a lymph node, a lymph vessel, an organ that is part of the lymphatic system or a combination thereof. In some aspects the therapeutic target is a joint. In some aspects, the therapeutic target is a lymph node. In some aspects, the therapeutic target is a specific lymph node as described elsewhere herein.

In some aspects the inflammatory locus is a joint selected from the group consisting of an ankle joint, a knee joint, a hip joint, a shoulder joint, an elbow joint, a metacarpophalangeal joint of the hands, a metatarsophalangeal joint in a foot, a wrist joint, a joint in the neck, and combinations thereof. In some aspects, the inflammatory locus is a psoriatic lesion.

In some aspects, the inflammatory locus is a knee, and the selected lymph capillaries and/or vessels flow into the popliteal lymph nodes. In some aspects, the inflammatory locus is a knee, and relative to the knee, the selected lymph capillaries and/or vessels are located distal to the heart.

In some aspects, the inflammatory locus is the neck, and the selected lymph capillaries and/or vessels flow into the cervical lymph nodes. In some aspects, the inflammatory locus is the neck, and, relative to the neck, the selected lymph capillaries and/or vessels are located distal to the heart.

In some aspects, the inflammatory locus is a shoulder, and the selected lymph capillaries and/or vessels flow into the pectoral lymph nodes, the superclavical lymph nodes, the axillary lymph nodes or any combination thereof. In some aspects, the inflammatory locus is a shoulder, and, relative to the shoulder, the selected lymph capillaries and/or vessels are located distal to the heart.

In some aspects, the inflammatory locus is an elbow, and the selected lymph capillaries and/or vessels flow into the epitrochlear lymph nodes and/or brachial lymph nodes. In some aspects, the inflammatory locus is an elbow, and, relative to the elbow, the selected lymph capillaries and/or vessels are located distal to the heart.

In some aspects, the inflammatory locus is a hip, and the selected lymph capillaries and/or vessels flow into the inguinal lymph nodes and/or the pelvic lymph nodes. In some aspects, the inflammatory locus is a hip, and, relative to the hip, the selected lymph capillaries and/or vessels are located distal to the heart. In some aspects, the inflammatory locus is a hip, and, relative to the hip, the selected lymph capillaries and/or vessels are located proximate to the heart.

In some aspects, the inflammatory locus is a psoriatic lesion and the selected lymph capillaries share common lymph vessels and/or lymph capillaries immediately adjacent to and/or within the psoriatic lesion. In some aspects, the medical device is placed at a location on the skin of the patient having lymph capillaries and/or vessels that flow directly into the lymph nodes within and/or closest to the psoriatic lesion.

In some aspects, when two medical devices that comprise a plurality of microneedles are used, the first medical device administers a first antibody that inhibits TNF-α or etanercept or a biosimilar or bioequivalent thereof to selected lymph capillaries and/or vessels distal to the heart relative to the inflammatory locus, and the second medical device administers a second antibody that inhibits TNF-α or etanercept or a biosimilar or bioequivalent thereof to selected lymph capillaries and/or vessels proximal to the heart relative to the inflammatory locus. In some aspects, the first therapeutic agent and the second therapeutic agent are the same. In some aspects, the first therapeutic agent and the second therapeutic agent are different. In this case each individual dose administered by each medical device may be smaller than a therapeutically effective dose, but the combined dose administered by the two medical devices is therapeutically effective.

In some embodiments, delivery of the antibody that inhibits TNF-α or etanercept or a biosimilar or bioequivalent thereof to the lymphatic system is delivery into the vessels of the lymphatic vasculature, the lymph nodes as described elsewhere herein, or both. In some aspects, delivery is to the superficial lymph vessels. In yet another aspect, delivery is to one or more lymph nodes. The specific target for delivery will be based on the medical needs of the patient. In one nonlimiting example, if a joint in the patient shows signs of an acute arthritic flare associated with a chronic arthritic condition, then the medical device comprising a plurality of microneedles can be placed on the patient such that it delivers the antibody that inhibits TNF-α or etanercept or a biosimilar or bioequivalent thereof directly to that specific joint. Alternatively, the medical device can be placed upstream of the joint such that the antibody that inhibits TNF-α or etanercept or a biosimilar or bioequivalent thereof is delivered to the lymph vessels that feed into the targeted joint. In some embodiments, two or more medical devices are used to target two or more different locations in the lymphatic system of the patient.

The placement of the medical device is based on the medical condition of the patient and/or an assessment by a medical professional. In one nonlimiting example, in a patient suffering from an acute flare-up of rheumatoid arthritis in one specific joint (e.g., the knee or shoulder), the medical device is placed upstream to deliver the agent to the lymph vessels that flow into and/or toward the inflamed joint in order to more effectively target the specific location of the acute flare-up. Similarly, in another nonlimiting example, a patient with significant patches of psoriatic lesions could have two or more medical devices placed in different locations on their body that are upstream of the lesions thereby targeting the specific lesions more precisely.

In some aspects, the antibody that inhibits TNF-α or etanercept or a biosimilar or bioequivalent thereof is effective in treating or relieving the symptoms of an inflammatory medical condition. In some embodiments, the antibody that inhibits TNF-α or etanercept or a biosimilar or bioequivalent thereof is adalimumab, adalimumab-atto, certolizumab pegol, etanercept, etanercept-szzs, golimumab, infliximab, infliximab-dyyb, or a biosimilar or bioequivalent of any of the foregoing agents. In some embodiments, the therapeutic agent is etanercept, a biosimilar thereof, or a bioequivalent thereof. In some embodiments, the therapeutic agent is adalimumab, a biosimilars thereof, or a bioequivalent thereof.

It is understood that when multiple doses of a therapeutic agent are administered to a patient, each individual dose may not be therapeutically effective, but the combined doses are therapeutically effective. The combined doses that are therapeutically effective may be smaller than a therapeutically effective dose if the same therapeutic agent is administered by a different route (e.g., subcutaneous, intravenous, etc.).

### VI. Medical Device

Medical devices that comprise an array of microneedles suitable for use herein are known in the art. Particular exemplary structures and devices comprising a means for controllably delivering one or more agents to a patient are described in International patent Application Publication Nos. WO 2014/188343, WO 2014/132239, WO 2014/132240, WO 2013/061208, WO 2012/046149, WO 2011/135531, WO 2011/135530, WO 2011/135533, WO 2014/132240, WO 2015/16821, and International Patent Applications PCT/US2015/028154 (published as WO 2015/168214 A1), PCT/US2015/028150 (published as WO 2015/168210 A1), PCT/US2015/028158 (published as WO 2015/168215 A1), PCT/US2015/028162 (published as WO 2015/168217 A1), PCT/US2015/028164 (published as WO 2015/168219 A1), PCT/US2015/038231 (published as WO 2016/003856 A1), PCT/US2015/038232 (published as WO 2016/003857 A1), PCT/US2016/043623 (published as WO 2017/019526 A1), PCT/US2016/043656 (published as WO 2017/019535 A1), PCT/US2017/027879 (published as WO 2017/189258 A1), PCT/US2017/027891 (published as WO 2017/189259 A1), PCT/US2017/064604 (published as WO 2018/111607 A1), PCT/US2017/064609 (published as WO 2018/111609 A1), PCT/US2017/064614 (published as WO 2018/111611 A1), PCT/US2017/064642 (published as WO 2018/111616 A1), PCT/US2017/064657 (published as WO 2018/111620 A1), and PCT/US2017/064668 (published as WO 2018/111621 A1), all of which are incorporated by reference herein in their entirety.

In some aspects of the embodiments described herein, the one or more therapeutic agents are administered by applying one or more medical devices to one or more sites of the skin of the patient. One nonlimiting example of a medical device comprising a plurality of microneedles that is suitable for use with all of the methods disclosed herein is the Sofusa™ drug delivery platform available from Sorrento Therapeutics, Inc.

In some embodiments, the medical device is placed in direct contact with the skin of the patient. In some embodiments, an intervening layer or structure will be between the skin of the patient and the medical device. For example, surgical tape or gauze may be used to reduce possible skin irritation between the medical device and the skin of the patient. When the microneedles extend from the apparatus, they will contact and, in some instances, penetrate the epidermis or dermis of the patient in order to deliver the medicament to the patient. The delivery of the medicament can be to the circulatory system, the lymphatic system, the interstitium, subcutaneous, intramuscular, intradermal or a combination thereof. In some embodiments, the medicament is delivered directly to the lymphatic system of the patient. In some aspects, the medicament is delivered to the superficial vessels of the lymphatic system.

The term “proximate” as used herein is intended to encompass placement on and/or near a desired therapeutic target. Placement of the medical device proximate to the therapeutic target results in the administered therapeutic agent entering the lymphatic system and traversing to the intended therapeutic target. Additionally, placement of the medical device may be such that the administered therapeutic agent is directly administered to the therapeutic target.

In some embodiments described herein, the methods comprising a medical device comprising a plurality of microneedles may comprise delivering one or more agents through a device comprising two or more delivery structures that are capable of penetrating the stratum corneum of the skin of a patient and obtaining a delivery depth and volume in the skin and controllably delivering one or more agents at the administration rates as described herein. The delivery structures may be attached to a backing substrate of the medical device and arranged at one or a plurality of different angles for penetrating the stratum corneum and delivering the one or more agents. In some aspects, described herein the backing substrate comprising the delivery structures may be in contact with the skin of a patient and may have a cylindrical, rectangular, or geometrically irregular shape. The backing substrate further comprises a two dimensional surface area that in some aspects may be from about 1 mm² to about 10,000 mm². In some aspects, the delivery structures may comprise any geometric shape (e.g., a cylindrical, rectangular or geometrically irregular shape). In addition, the delivery structures may comprise a length and cross sectional surface area. In some aspects, the delivery structures may have an overall length that is greater than a cross sectional diameter or width. In some other aspects, the delivery structures may have a cross sectional diameter or width greater than an overall length. In some aspects, the cross sectional width of each of the delivery structures may be from about 5 μm to about 140 μm and the cross sectional area may be from about 25 μm² to about 65,000 μm², including each integer within the specified range. In some embodiments, the length of each of the delivery structures may be from about 10 μm to about 5,000 μm, from about 50 to about 3,000 μm, from about 100 to about 1,500 μm, from about 150 to about 1,000 μm, from about 200 to about 800 μm, from about 250 to about 750 μm, or from about 300 to about 600 μm. In some aspects, the length of each of the delivery structures may be from about 10 μm to about 1,000 μm, including each integer within the specified range. The surface area and cross-sectional surface areas as described herein may be determined using standard geometric calculations known in the art.

The delivery structures described herein need not be identical to one another. A medical device having a plurality of delivery structures may each have various lengths, outer diameters, inner diameters, cross-sectional shapes, nanotopography surfaces, and/or spacing between each of the delivery structures. For example, the delivery structures may be spaced apart in a uniform manner, such as, for example, in a rectangular or square grid or in concentric circles. The spacing may depend on numerous factors, including height and width of the delivery structures, as well as the amount and type of an agent that is intended to be delivered through the delivery structures. In some aspects, the spacing between each delivery structure may be from about 1 μm to about 1500 μm, including each integer within the specified range. In some aspects, the spacing between each deliver structure may be about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm or about 1500 μm. About as used in this context, “about” means±50 μm.

In some embodiments described herein, the medical device may comprise a needle array in the form of a patch. In some aspects, the array of needles are able to penetrate a most superficial layer of the stratum corneum and initially deliver one or more agents as described herein to at least a portion or all of the non-viable epidermis, at least a portion of or all of the viable epidermis, and/or at least a portion of the viable dermis of a subject and subsequently to the lymphatic system of the patient. These needles may further comprise nanotopography on the surface of the needle in a random or organized pattern. In some aspects, the nanotopography pattern may demonstrate fractal geometry.

In some embodiments, the delivery structures may comprise an array of needles in fluid connection with a liquid carrier vehicle comprising one or more agents. In some aspects, the needles are microneedles. In some aspects, the array of needles may comprise between 2 and 50,000 needles with structural means for controlling skin penetration and fluid delivery to the skin (e.g., penetrating and delivering to the skin), see e.g., International Patent Application PCT/US2017/064668 (published as WO 2018/111621 A1), which is incorporated by reference herein in its entirety. In some other aspects, the array of needles may further comprise a manufactured random or structured nanotopography on each needle. The needle or needle array may be attached to a larger drug delivery apparatus comprising fluidic delivery rate controls, adhesives for attaching to the skin, fluidic pumps, and the like. If desired, the rate of delivery of the agent may be variably controlled by the pressure-generating means. Desired delivery rates as described herein to the epidermis may be initiated by driving the one or more agents described herein with the application of pressure or other driving means, including pumps, syringes, pens, elastomer membranes, gas pressure, piezoelectric, electromotive, electromagnetic or osmotic pumping, or use of rate control membranes or combinations thereof.

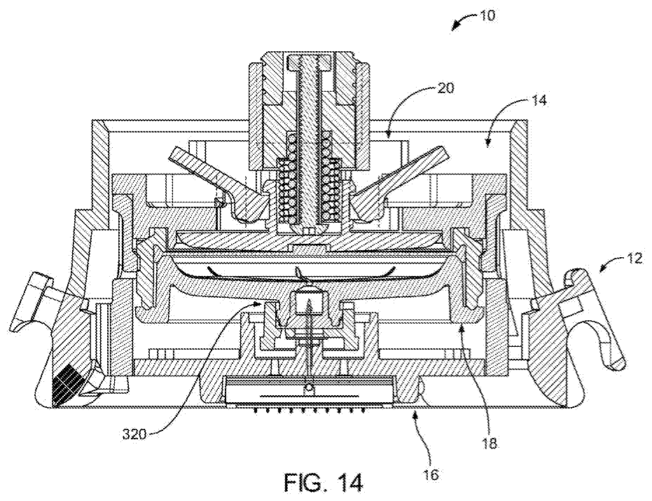

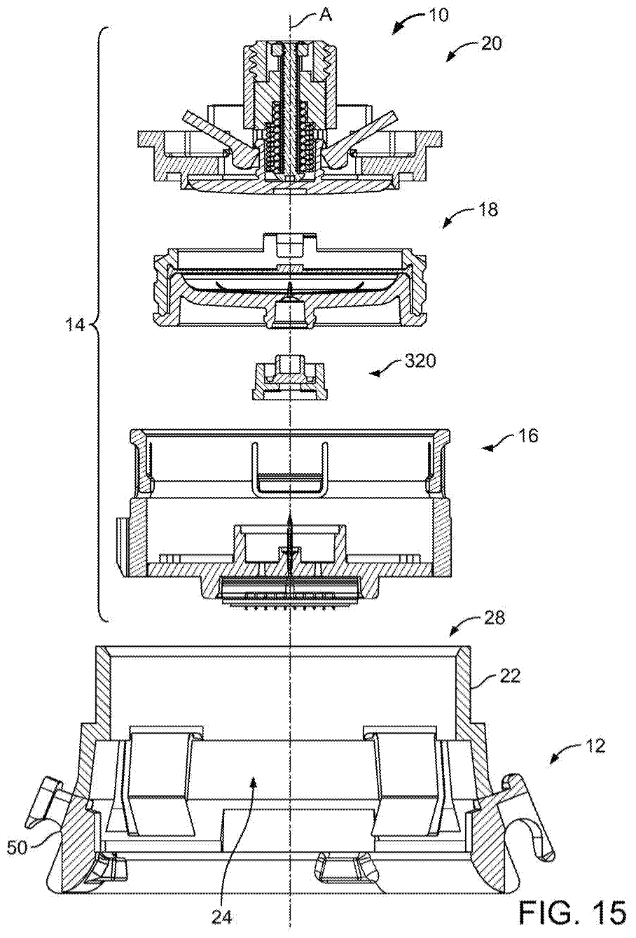

FIG. 13 is a sectional view of one exemplary example of a medical device comprising a plurality of microneedles (e.g., a medicament delivery apparatus), indicated generally by 10, in a pre-use configuration. It is understood that this example is suitable for use with all embodiments and aspects of the subject matter disclosed herein. Other devices as are known in the art are also suitable for use herewith. FIG. 14 is a sectional view of the fluid delivery apparatus 10 in a use configuration. FIG. 15 is an exploded, sectional view of fluid delivery apparatus 10. In the exemplary embodiment, the fluid delivery apparatus 10 includes a plurality of subassembly components coupled together to form the fluid delivery apparatus 10, including a collet assembly 12 and a fluid distribution assembly 14. The collet assembly 12 and the fluid distribution assembly 14 are indicated generally by their respective reference numbers. As shown in FIG. 15, the fluid distribution assembly 14 includes a plurality of additional subassembly components, including a plenum assembly 16, a cartridge assembly 18, a cap assembly 320, and a mechanical controller assembly 20. Each of the collet assembly 12, the fluid distribution assembly 14, the plenum assembly 16, the cartridge assembly 18, the cap assembly 320, and the mechanical controller assembly 20 is indicated generally in the accompanying drawings by their reference numbers. The collet assembly 12 forms the body or housing of the fluid delivery apparatus 10 and is slidably coupled to the fluid distribution assembly 14. To form the fluid distribution assembly 14, the cap assembly 320 is coupled to the cartridge assembly 18, and the cartridge assembly 18 is slidably coupled to the plenum assembly 16. In addition, the mechanical controller assembly 20, as explained in more detail below, is coupled to the cartridge assembly 18.

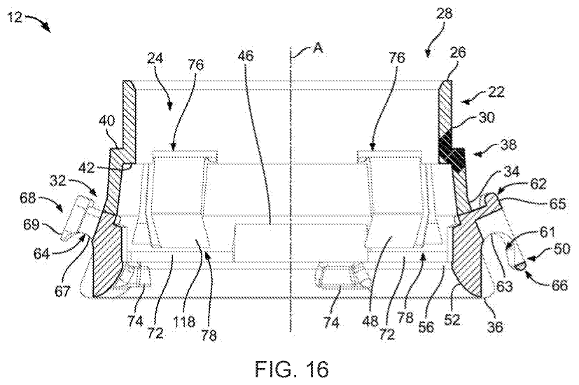

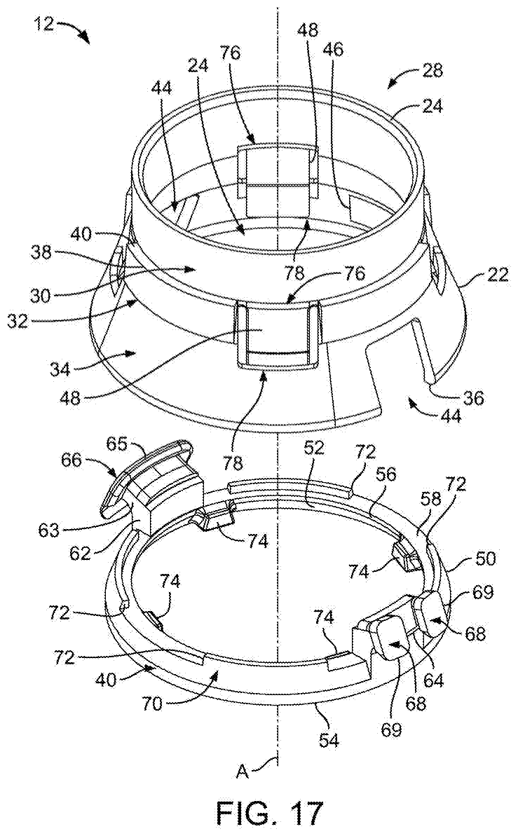

FIG. 16 is a sectional view and FIG. 17 is an exploded, perspective of the collet assembly 12 of the fluid delivery apparatus 10. Referring to FIGS. 15-17, in the exemplary embodiment, the collet assembly 12 includes a collet 22 coupled to a collet lock 50. In the exemplary embodiment, the collet 22 is formed in a generally frustoconical shape, having a hollow interior space 24 defined therein. The collet 22 is formed generally symmetrically about a central axis “A.” An upper rim 26 of the collet 22 defines an opening 28 to the interior space 24. A cylindrical upper wall 30 extends generally vertically downward from the upper rim 26 towards a central portion 32 of the collet 22. A lower wall 34 extends downward at an outward angle from the central portion 32 toward a base 36 (or lower edge) of the collet 22. The upper wall 30, central portion 32, and the lower wall 34 collectively define the interior space 24. A step 38 extends around the upper wall 30, defining an outer horizontal surface 40 (or ledge) configured to engage an attachment band. The step 38 also defines an inner horizontal surface 42 (or step) configured to engage with the plenum assembly 16 to facilitate properly positioning the plenum assembly 16 above a user's skin surface prior to use of the fluid delivery apparatus 10.

As illustrated in FIG. 17, the collet 22 includes a pair of notches, indicated generally at 44, opposite each other and formed through the lower wall 34. In the exemplary embodiment, the notches 44 are generally rectangular in shape and configured to receive a portion of the collet lock 50. In addition, the collet 22 includes one or more stops 46 configured to facilitate positioning of the collet lock 50 when coupled to the collet 22. For example, and without limitation, the one or more stops 46 are formed as inward extending projections formed on lower wall 34. The stops 46 can have form or shape that enables the stops 46 to function as described herein.

As illustrated in FIGS. 16 and 17, the collet 22 includes a plurality of flexible tabs 48 formed integrally with the upper wall 30. In addition, the plurality of flexible tabs 48 is positioned about and equidistant from the central axis “A.” In particular, the plurality of flexible tabs 48 extends from a first end 76 to an opposite free second end 78. In the exemplary embodiment, the free second end 78 angles radially inward and is configured to engage with the plenum assembly 16 to facilitate properly positioning the plenum assembly 16 at the user's skin surface during use of the fluid delivery apparatus 10.

As illustrated in FIGS. 16 and 17, in the exemplary embodiment, the collet lock 50 is generally ring-shaped, having a convex inner surface 52 extending from a lower outer edge 54 of the collet lock 50 to a generally cylindrical inner wall 56. The inner wall 56 extends upward to an upper surface 58. The collet lock 50 includes a generally cylindrical outer wall 60 that is concentric with inner wall 56 and extends upward from the lower outer edge 54. In addition, the collet lock 50 includes latching members 62, 64, opposite each other and extending upward from the upper surface 58. The latching members 62, 64 are configured to couple to the notches 44 of the collet 22. The latch member 62 includes a first coupling member 66 that extends outward from latch member 62. In particular, the first coupling member 66 includes a neck portion 63 that extends at an upward angle substantially perpendicular to the lower wall 34 of the collet 22. In addition, the first coupling member 66 includes a head portion 65 that extends generally parallel to the lower wall 34 beyond a periphery of the neck portion 63. Furthermore, the first coupling member 66 includes a window or aperture 61 extending through the head portion 65. The window 61 is configured to present an indication to the user of the fluid delivery apparatus 10 of a tightness of the attachment band 430, as is further described herein.

Similarly, the latching member 64 includes an adjacent pair of second coupling members 68 that extend outward from latching member 64. In the exemplary embodiment, the coupling members 68 each include a neck portion 67 that extends at an upward angle substantially perpendicular to the lower wall 34 of the collet 22. In addition, the second coupling members 68 include a head portion 69 that extends generally parallel to the lower wall 34 beyond a periphery of the neck portion 67. The first coupling member 66 and the pair of second coupling members 68 are configured to engage the attachment band 430, as is described further herein.

In the exemplary embodiment, the outer wall 60 of the collet lock 50 includes an upper outer surface 70 that inclines inward at an angle substantially parallel to the lower wall 34 to facilitate face-to-face engagement therewith. In addition, the upper surface 58 includes a plurality of stop members 72 that extend upward and are configured to engage the one or more stops 46 of the collet 22 to facilitate properly positioning of the collet lock 50 when coupled to the collet 22. Extending radially inward from the convex inner surface 52 is a plurality of tabs 74 configured to engage with the plenum assembly 16 to facilitate properly positioning the plenum assembly 16 at the user's skin surface during use of the fluid delivery apparatus 10.

In the exemplary embodiment, the collet 22 is coupled to the collet lock 50 to form a unitary assembly (shown in FIG. 16). In particular, the upper surface 70 and the latching members 62, 64 of the collet lock 50 engage the lower wall 34 and the notches 44 of the collet 22 via a permanent coupling method, for example, and without limitation, via an adhesive bond, a weld joint (e.g., spin welding, ultrasonic welding, laser welding, or heat staking), and the like. Alternatively, the collet 22 and the collet lock 50 may be coupled together using any connection technique that enables the formation of the collet assembly 12.

Additional description of the fluid delivery apparatus 10 seen FIGS. 13-17, including its operation, can be found in PCT/US2017/064668 (published as WO 2018/111621 A1), which is hereby incorporated by reference in its entirety.

In some embodiments described herein, medical devices comprising a plurality of microneedles as described herein functions as a permeability enhancer and may increase the delivery of one or more agents through the epidermis. This delivery may occur through modulating transcellular transport mechanisms (e.g., active or passive mechanisms) or through paracellular permeation. Without being bound by any theory, the nanostructured or nanotopography surface may increase the permeability of one or more layers of the viable epidermis, including the epidermal basement membrane by modifying cell/cell tight junctions allowing for paracellular or modifying cellular active transport pathways (e.g., transcellular transport) allowing for diffusion or movement and/or active transport of an administered agent through the viable epidermis and into the underlying viable dermis. This effect may be due to modulation of gene expression of the cell/cell tight junction proteins. As previously mentioned, tight junctions are found within the viable skin and in particular the viable epidermis. The opening of the tight junctions may provide a paracellular route for improved delivery of any agent, such as those that have previously been blocked from delivery through the skin.

Interaction between individual cells and structures of the nanotopography may increase the permeability of an epithelial tissue (e.g., the epidermis) and induce the passage of an agent through a barrier cell and encourage transcellular transport. For instance, interaction with keratinocytes of the viable epidermis may encourage the partitioning of an agent into the keratinocytes (e.g., transcellular transport), followed by diffusion through the cells and across the lipid bilayer again. In addition, interaction of the nanotopography structure and the corneocytes of the stratum corneum may induce changes within the barrier lipids or corneodesmosomes resulting in diffusion of the agent through the stratum corneum into the underlying viable epidermal layers. While an agent may cross a barrier according to paracellular and transcellular routes, the predominant transport path may vary depending upon the nature of the agent.

In some embodiments described herein, the device may interact with one or more components of the epithelial tissue to increase porosity of the tissue making it susceptible to paracellular and/or transcellular transport mechanisms. Epithelial tissue is one of the primary tissue types of the body. Epithelial tissues that may be rendered more porous may include both simple and stratified epithelium, including both keratinized epithelium and transitional epithelium. In addition, epithelial tissue encompassed herein may include any cell types of an epithelial layer including, without limitation, keratinocytes, endothelial cells, lymphatic endothelial cells, squamous cells, columnar cells, cuboidal cells and pseudostratified cells. Any method for measuring porosity may be used including, but not limited to, any epithelial permeability assay. For example, a whole mount permeability assay may be used to measure epithelial (e.g., skin) porosity or barrier function in vivo see, for example, Indra and Leid, Methods Mol Biol. (763) 73-81, which is incorporated by reference herein for its teachings thereof.

In some embodiments described herein, the structural changes induced by the presence of a nanotopography surface on a barrier cell are temporary and reversible. It was surprisingly found that using nanostructured nanotopography surfaces results in a temporary and completely reversible increase in the porosity of epithelial tissues by changing junctional stability and dynamics, which, without being bound by any theory, may result in a temporary increase in the paracellular and transcellular transport of an administered agent through the epidermis and into the viable dermis. Thus, in some aspects, the increase in permeability of the epidermis or an epithelial tissue elicited by the nanotopography, such as promotion of paracellular or transcellular diffusion or movement of one or more agents, returns to a normal physiological state that was present before contacting the epithelial tissue with a nanotopography following the removal of the nanotopography. In this way, the normal barrier function of the barrier cell(s) (e.g., epidermal cell(s)) is restored and no further diffusion or movement of molecules occurs beyond the normal physiological diffusion or movement of molecules within the tissue of a subject.

These reversible structural changes induced by the nanotopography may function to limit secondary skin infections, absorption of harmful toxins, and limit irritation of the dermis. Also, the progressive reversal of epidermal permeability from the top layer of the epidermis to the basal layer may promote the downward movement of one or more agents through the epidermis and into the dermis and prevent back flow or back diffusion of the one or more agents back into the epidermis.

In some embodiments described herein, are methods for applying a device having a plurality of microneedles to the surface of the skin a subject for the treatment of a disease or disorder described herein. In some aspects, the device is applied to an area of the subject's skin, wherein the location of the skin on the body is dense in lymphatic capillaries and/or blood capillaries. Multiple devices may be applied to one or more locations of the skin having a dense network of lymphatic capillaries. In some aspects, 1, 2, 3, 4, 5, or more devices may be applied. These devices may be applied spatially separate or in close proximity or juxtaposed with one another. Exemplary and non-limiting locations dense with lymphatics comprise the palmar surfaces of the hands, the scrotum, the plantar surfaces of the feet and the lower abdomen. The location of the device will be selected based on the medical condition of the patient and the assessment of a medical professional.

In some embodiments described herein, at least a portion of or all of the therapeutic agent may be directly delivered or administered to an initial depth in the skin comprising the nonviable epidermis and/or the viable epidermis. In some aspects, a portion of therapeutic agent may also be directly delivered to the viable dermis in addition to the epidermis. The range of delivery depth will depend on the medical condition being treated and the skin physiology of a given patient. This initial depth of delivery may be defined as a location within the skin, wherein a therapeutic agent first comes into contact as described herein. Without being bound by any theory, it is thought that the administered agent may move (e.g., diffuse) from the initial site of delivery (e.g., the non-viable epidermis, the viable epidermis, the viable dermis, or the interstitium) to a deeper position within the viable skin. For example, a portion of or all of an administered agent may be delivered to the non-viable epidermis and then continue to move (e.g., diffuse) into the viable epidermis and past the basal layer of the viable epidermis and enter into the viable dermis. Alternatively, a portion of or all of an administered agent may be delivered to the viable epidermis (i.e., immediately below the stratum corneum) and then continue to move (e.g., diffuse) past the basal layer of the viable epidermis and enter into the viable dermis. Lastly, a portion of or all of an administered agent may be delivered to the viable dermis. The movement of the one or more active agents throughout the skin is multifactorial and, for example, depends on the liquid carrier composition (e.g., viscosity thereof), rate of administration, delivery structures, etc. This movement through the epidermis and into the dermis may be further defined as a transport phenomenon and quantified by mass transfer rate(s) and/or fluid mechanics (e.g., mass flow rate(s)).

Thus, in some embodiments described herein, the therapeutic agent may be delivered to a depth in the epidermis wherein the therapeutic agent moves past the basal layer of the viable epidermis and into the viable dermis. In some aspects described herein, the therapeutic agent is then absorbed by one or more susceptible lymphatic capillary plexus then delivered to one or more lymph nodes and/or lymph vessels.

In some embodiments described herein, the distribution of depths in the skin, wherein a portion of the one or more agents is initially delivered, which results in uptake of the one or more therapeutic agents by one or more susceptible tumors or inflammatory locus, or by lymph vessels that feed into the tumors or inflammatory locus, ranges from about 5 μm to about 4,500 μm. Because the thickness of the skin can vary from patient to patient based on numerous factors, including, but not limited to, medical condition, diet, gender, age, body mass index, and body part, the required depth to deliver the therapeutic agent will vary. In some aspects, the delivery depth is from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm. In some aspects, the delivery depth is about 50 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, or about 1000 μm. As used in this context, “about” means±50 μm.

In some embodiments described herein, the therapeutic agent may be delivered in a liquid carrier solution. In one aspect, the tonicity of the liquid carrier may be hypertonic to the fluids within the blood capillaries or lymphatic capillaries. In another aspect, the tonicity of a liquid carrier solution may be hypotonic to the fluids within the blood capillaries or lymphatic capillaries. In another aspect, the tonicity of a liquid carrier solution may be isotonic to the fluids within the blood capillaries or lymphatic capillaries. The liquid carrier solution may further comprise at least one or more pharmaceutically acceptable excipients, diluent, cosolvent, particulates, or colloids. Pharmaceutically acceptable excipients for use in liquid carrier solutions are known, see, for example, Pharmaceutics: Basic Principles and Application to Pharmacy Practice (Alekha Dash et al. eds., 1st ed. 2013), which is incorporated by reference herein for its teachings thereof.

In some embodiments described herein, the therapeutic agent is present in a liquid carrier as a substantially dissolved solution, a suspension, or a colloidal suspension. Any suitable liquid carrier solution may be utilized that meets at least the United States Pharmacopeia (USP) specifications, and the tonicity of such solutions may be modified as is known, see, for example, Remington: The Science and Practice of Pharmacy (Lloyd V. Allen Jr. ed., 22nd ed. 2012. Exemplary non-limiting liquid carrier solutions may be aqueous, semi-aqueous, or nonaqueous depending on the bioactive agent(s) being administered. For example, an aqueous liquid carrier may comprise water and any one of or a combination of a water-miscible vehicles, ethyl alcohol, liquid (low molecular weight) polyethylene glycol, and the like. Non-aqueous carriers may comprise a fixed oil, such as corn oil, cottonseed oil, peanut oil, or sesame oil, and the like. Suitable liquid carrier solutions may further comprise any one of a preservative, antioxidant, complexation enhancing agent, a buffering agent, an acidifying agent, saline, an electrolyte, a viscosity enhancing agent, a viscosity reducing agent, an alkalizing agent, an antimicrobial agent, an antifungal agent, a solubility enhancing agent or a combination thereof.

In some embodiments described herein, the therapeutic agent is delivered to the viable skin, wherein the distribution of depths in the viable skin for delivery of the agent is immediately past the stratum corneum of the epidermis but above the subcutaneous tissue, which results in uptake of the agent by the lymphatic vasculature of the patient. In some aspects, the depth in the viable skin for delivering one or more agents ranges from about 1 μm to about 4,500 μm beyond the stratum corneum, but still within the viable skin above the subcutaneous tissue.

Non-limiting tests for assessing initial delivery depth in the skin may be invasive (e.g., a biopsy) or non-invasive (e.g., imaging). Conventional non-invasive optical methodologies may be used to assess delivery depth of an agent into the skin including remittance spectroscopy, fluorescence spectroscopy, photothermal spectroscopy, or optical coherence tomography (OCT). Imaging using methods may be conducted in real-time to assess the initial delivery depths. Alternatively, invasive skin biopsies may be taken immediately after administration of an agent, followed by standard histological and staining methodologies to determine delivery depth of an agent. For examples of optical imaging methods useful for determining skin penetration depth of administered agents, see, Sennhenn et al., Skin Pharmacol. 6(2) 152-160 (1993), Gotter et al., Skin Pharmacol. Physiol. 21 156-165 (2008), or Mogensen et al., Semin. Cutan. Med. Surg 28 196-202 (2009), each of which are incorporated by reference herein for their teachings thereof.

In some embodiments described herein are methods for the extended delivery (or administration) of the therapeutic agent as described herein. The medical device comprising a plurality of microneedles is configured such that that the flow rate of the medicament from the device into the patient can be adjusted. As such, the length of time required will vary accordingly. In some aspects, the flow rate of the medical device is adjusted such that the medicament is administered over from about 0.5 hours to about 72 hours. In some aspects the time period for administration is about 1 hour, 2 hours, 3 hours, 4 hours, 5 hours, 6 hours, 7 hours, 8 hours, 9 hours, 10 hours, 12 hours, 15 hours 18 hours, 21 hours, 24 hours, 27 hours, 30 hours, 33 hours, 36 hours, 39 hours, 42 hours, 45 hours, 48 hours, 51 hours, 54 hours, 57 hours, 60 hours, 63 hours, 66 hours, 69 hours or 72 hours. In other aspects, the time period for administration is selected based on the medical condition of the patient and an assessment by the medical professional treating the patient.

In some embodiments described herein, one or more agents in a liquid carrier solution are administered to an initial approximate volume of space below the outer surface of the skin. The one or more therapeutic agents in a liquid carrier solution initially delivered to the skin (e.g., prior to any subsequent movement or diffusion) may be distributed within, or encompassed by an approximate three dimensional volume of the skin. The one or more initially delivered agents exhibits a Gaussian distribution of delivery depths and will also have a Gaussian distribution within a three dimensional volume of the skin tissue.

In some embodiments described herein, the flow rate of the therapeutic agent to the skin per single microneedle as described herein may be about 0.01 μl per hour to about 500 μl per hour. In some aspects, the flow rate for each individual microneedle is from about 0.1 μl per hour to about 450 μl per hour, about 0.5 μl per hour to about 400 μl per hour, about 1.0 μl per hour to about 350 μl per hour, about 5.0 μl per hour to about 300 μl per hour, about 5.0 μl per hour to about 250 μl per hour, about 10 μl per hour to about 200 μl per hour, about 15 μl per hour to about 100 μl per hour, or about 20 μl per hour to about 50 μl per hour. In some aspects, the flow rate for each individual microneedle is about 1 μl per hour, 2 μl per hour, 5 μl per hour, 10 μl per hour, 15 μl per hour, 20 μl per hour, 25 μl per hour, 30 μl per hour, 40 μl per hour, 50 μl per hour, 75 μl per hour, or 100 μl per hour. Each individual microneedle will have a flow rate that contributes to the overall device flow rated. The maximum overall flow rate will be flow rate of each individual microneedle multiplied by the total number of microneedles. The overall controlled flow rate of all of the combined microneedles may be from about 0.2 μl per hour to about 50,000 μl per hour. The medical device is configured such that that the flow rate can be controlled appropriately. The flow rate will be based upon the medical condition of the patient and an assessment by the medical professional treating the patient.

### EXAMPLES

### Example 1—Etanercept Pharmacokinetics

Methods and procedures for some of these experiments have been adapted from Aldrich, et al., Arthritis Res. Ther., (2017), 19:116 (DOI 10.1186/s13075-017-1323-z; Open Access) which is incorporated by reference herein in its entirety for all purposes.

In order to characterize differences in etanercept delivery by the different routes of administration, normal rats were used to determine the PK profiles following a single dose of 1 mg etanercept in 100 μL delivered via Sofusa™ (n=6), conventional IV (in the tail vein, n=6), SC (dorsolateral injection at the same site as the Sofusa™ application, n=4), or ID injections (symmetrical dorsolateral injections 2×50 uL for a total dose of 1 mg etanercept, n=6). Other than the Sofusa™ delivery, all administration was conducted using a 31-gauge needle. At 2, 4, 8, 12, 24, and 36 h after administration, animals were anesthetized under isoflurane, and 200 μL of blood was drawn from the jugular vein. The etanercept concentration in serum was quantified using the Etanercept ELISA Kit (ABIN: 1540251) (Matriks Biotechnology Co., LTD., Ankara, Turkey). Optical density was measured at 450 nm using Thermo Scientific Multiskan EX (Thermo Fisher Scientific, Waltham, Mass., USA).

In order to determine the amount etanercept delivered to the lymph nodes, a radiolabeled etanercept solution was administrated using the Sofusa™ drug delivery platform (1 mg etanercept in 100 μL), IV (1 mg etanercept in the tail vein in 100 μL), SC (1 mg etanercept in the dorsolateral side at the same site as the Sofusa™ application, 100 μL), and ID (1 mg total etanercept in two 50 μL injections). Animals were harvested at 12 and 36 hrs after administration, and the left/right axillary and inguinal lymph nodes were collected, weighed, and counted for radioactivity using a 2480 Wizard2 automatic gamma counter (PerkinElmer, Waltham, Mass., USA). The time-corrected radioactivity was then used to compute the μg/mL of tissue etanercept concentration from the specific radioactivity of the dosing solution with animals that were euthanized at 12 hrs (n=4, Sofusa™; n=6, IV; n=6, ID; and n=6, SC) and at 36 hrs (n=6, Sofusa™; n=6, IV; n=6, ID; and n=6, SC).

In order to validate the amount of etanercept delivered, the radioactivity was measured in the Sofusa™ device and tubing before and after the 1 hr infusion. Measurement was conducted using the dose calibrator. The amount of etanercept delivered as the difference in the time-corrected radioactivity before and after administration was determined.

To directly visualize Sofusa™ delivery, 100 μL of 645 μM indocyanine green (ICG) (Akorn, Inc.) in sterile saline was delivered over 1 hr using a syringe pump (model NE-300, SyringePump.com) connected to the Sofusa™, which was applied to the dorsal surface on the right side of the rat. NIRF imaging was conducted.

In vivo testing of the bioavailability and biodistribution of etanercept in both the rat and porcine model showed significantly superior pharmacokinetic profiles when compared to subcutaneous injection and are comparable to intravenous administration in many aspects.

Results in Sprague Dawley Rats

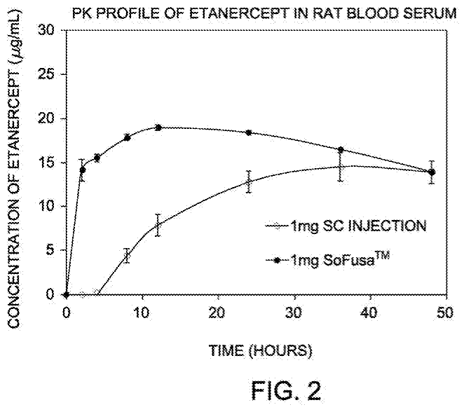

As summarized in Tables 1 and 2 and shown in FIG. 2, subcutaneous (SC) administration of etanercept gave significantly inferior results when compared to administration via the Sofusa™ drug delivery platform. The Tₘₐₓ was significantly shorter while the Cₘₐₓ was longer. The AUC₄₈ was almost double when using the Sofusa™ drug delivery platform. Significantly, the bioavailability (BA) increased from 17% to 29%.

TABLE 1
Pharmacokinetic data for etanercept in a rat model

| Delivery | | Tₘₐₓ | Cₘₐₓ | AUC₀₋₄₈ | BA |
|---|---|---|---|---|---|
| Technology | N | (hrs) | (μg/mL) | (hrs-μg/mL) | (%) |
| IV | 6 | 8 | 120 | 2,800 | 100 |
| SC | 6 | 36 | 15 | 302 | 10 |
| Sofusa ™ Study 1 | 12 | 4 | 25 | 545 | 20 |
| Sofusa ™ Study 2 | 6 | 2 | 22 | 502 | 18 |
| Sofusa ™ Study 3 | 6 | 2 | 20 | 538 | 19 |

TABLE 2
Summary of pharmacokinetic of etanercept SC vs the Sofusa ™ drug
delivery platform in a rat model.

| Delivery | Tₘₐₓ | Cₘₐₓ | AUC₀₋₄₈ | BA |
|---|---|---|---|---|
| Technology | (hrs) | (μg/mL) | (hrs-μg/mL) | (%) |
| SC | 36 | 15 | 492 | 17 |
| Sofusa ™ | 10 | 19 | 800 | 29 |

TABLE 3
Summary of the PK/PD data in the rat model.

| Route of | Cₘₐₓ | Tₘₐₓ | AUC₀₋₃₆ | BA |
|---|---|---|---|---|
| Administration | (μg/ml) | (hour) | (μg-hour/ml) | (%) |
| Subcutaneous | 15 | 36 | 302 | 11 |
| Intradermal | 21 | 12 | 696 | 25 |
| Sofusa ™ | 23 | 12 | 1010 | 36 |
| Intravenous | 120 | 2 | 2,815 | 100 |

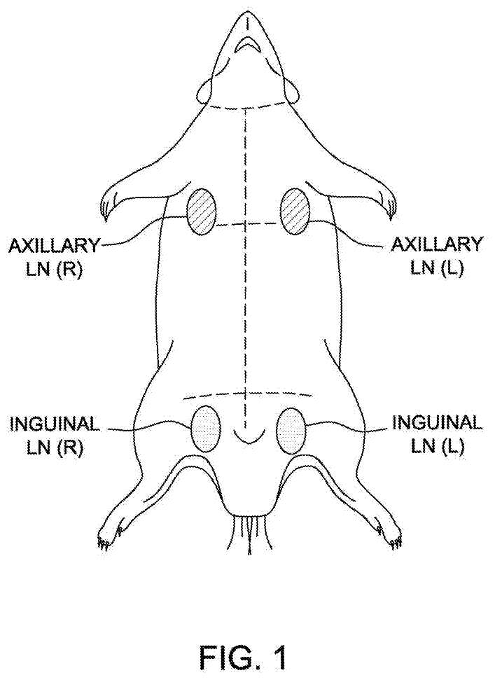

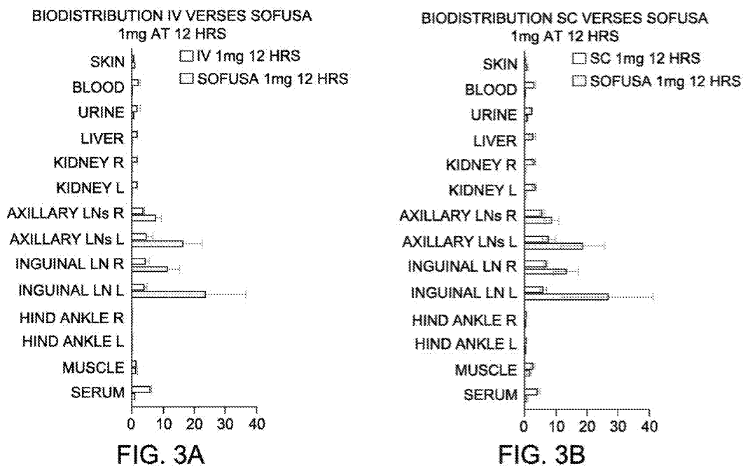

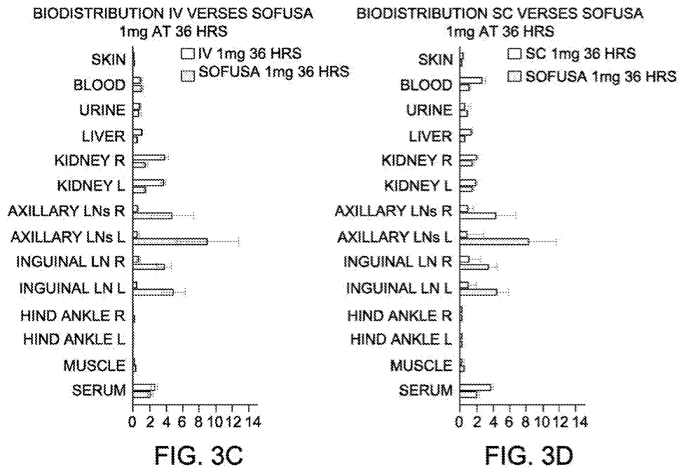

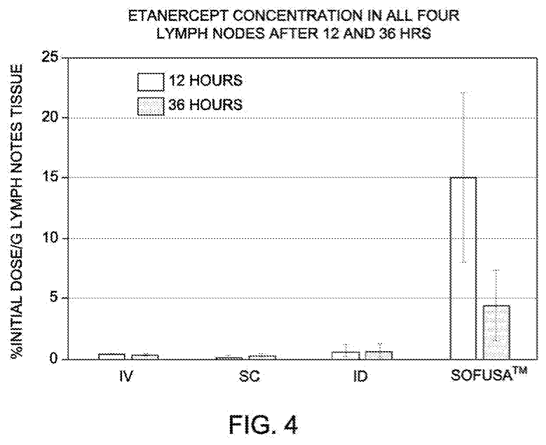

FIG. 1 illustrates the location of key lymph nodes in the rat that were examined in this experiment. FIGS. 3A to 3D illustrate a comparison of the biodistribution at both 12 and 36 hours of etanercept in vivo when administered using Sofusa™ as compared to intravenous and subcutaneous injection methods. Using the Sofusa™ drug delivery platform resulted in significantly more of the active agent in the axillary and inguinal lymph nodes as compared to either of the other methods of administration. Such targeted administration may be able to significantly reduce metastasis in cancer that has begun spreading to the lymph nodes. FIGS. 3A to 3D illustrate the difference in the biodistribution of etanercept when administered using the Sofusa™ drug delivery platform when compared to intravenous (FIGS. 3A and 3C) or subcutaneous (FIG. 3B or 3D) administration. At both 12 and 36 hours, when administered using the Sofusa™ drug delivery platform, there is a much larger proportion of etanercept located in the axillary and inguinal lymph nodes of the rat compared to the other methods of administration. FIG. 4 combines and summarizes the lymph node distribution data from FIGS. 3A to 3D and clearly illustrates the significant change in the biodistribution pattern of etanercept when administered using the Sofusa™ drug delivery platform. For tumors that exhibit a high risk of metastasis, this would greatly decrease the chance of the cancer spreading which would lead to a better patient outcome.

Collagen Induced Arthritic (CIA) Model for Rheumatoid Arthritis (RA)

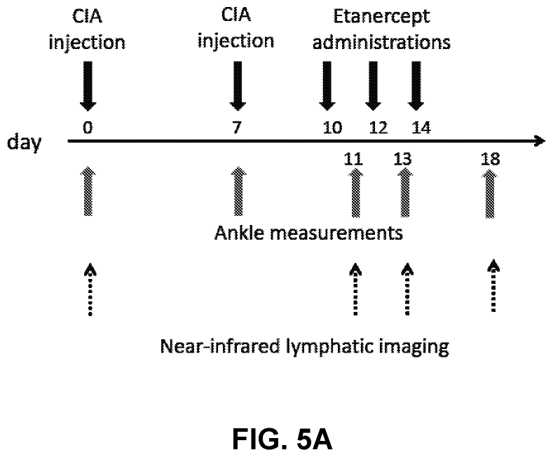

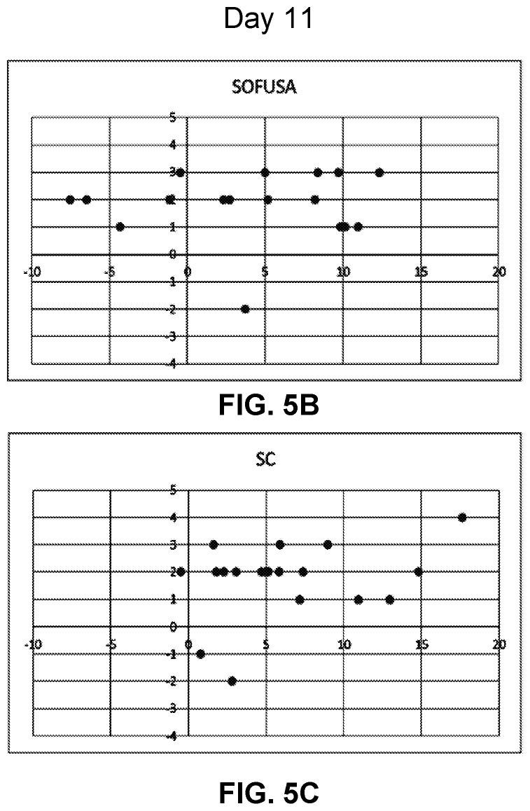

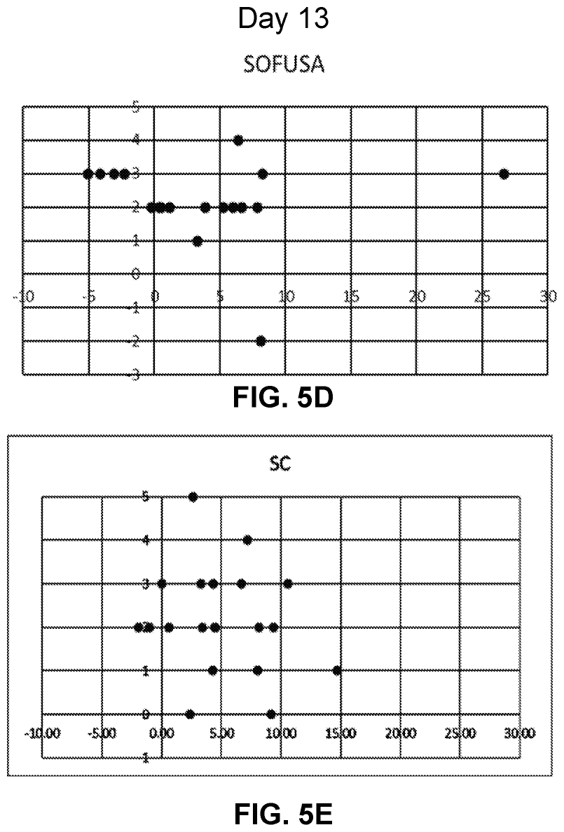

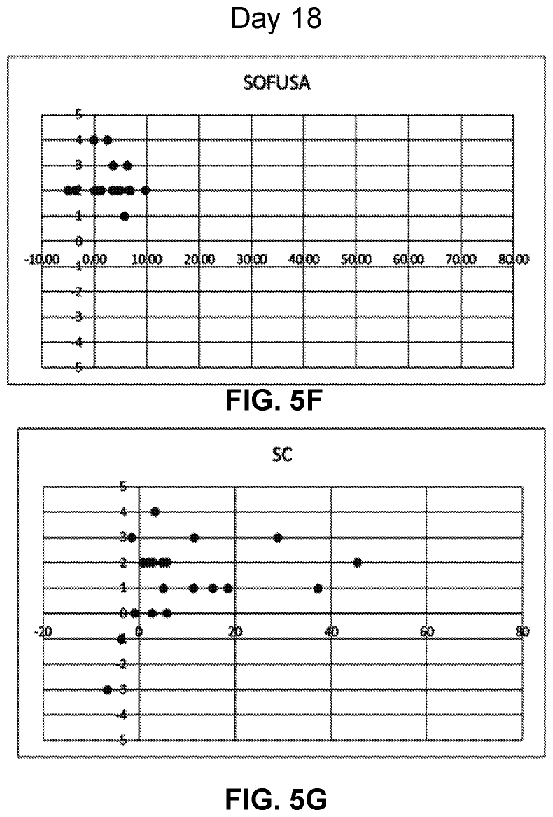

FIG. 5A shows the timeline for induction, measurement, and treatment of CIA animals. Specifically, type II porcine collagen (Chondrex, Inc. catalog #20031), solubilized in 0.05 N of acetic acid in sterile water at a concentration of 2 mg/mL was emulsified with an equal volume of incomplete Freud's adjuvant (Chondrex, Inc. catalog #7002) using homogenization at 35,000 rpm (Omni International homogenizer TH, homogenizer probe #32750); 100 μL emulsion was injected subcutaneously at the base of the tails on both sides for initial administration (day 0, 200 μL of emulsion total), and then again, 7 days later, on the right side only, for booster administration (day 7, 100 μL of emulsion total). Hind limb swelling, usually evident by day 14, was assessed by caliper measurements of the rear ankle cross (side-to-side) and oblique (front-to-back) dimensions. The two measurements for each hind limb were multiplied together for assessment of swelling, and the percent change from baseline was computed. These measurements and lymphatic imaging were performed on days 0, 7, 11, 13, and 18, in the early stages of CIA before the onset of joint destruction. Four groups of animals were studied: (1) untreated (n=20), (2) treated with etanercept given by SC administration (n=20, 1 mg in 100 μL), (3) treated with etanercept given by ID administration (n=20, two administrations of 0.5 mg in 50 μL), and (4) treated with etanercept given via Sofusa™ (n=18, one administration of 1 mg in 100 μL delivered).

Lymphatic Pumping Rate

Rats were anesthetized with isoflurane and shaved before imaging; 10 μL of 625 μM ICG was then injected ID with a 31-gauge needle/syringe (BD #328438, Fisher Scientific) at the base of the tail and on the dorsal side of the paw on both the right and left sides of the rats to perform NIRF imaging of the lymphatics. NIRF images were collected with a custom-built system that employed illumination of tissue surfaces with 785 nm light from a laser diode (85 mA and 80 mW, DL7140-201, Sanyo) that was diffused to cover a circular area approximately 8 cm in diameter. Fluorescent light generated from the ICG within the lymphatic vasculature was collected with an electron-multiplying charge-coupled device (EMCCD) (model 7827-0001, Princeton Instruments). Filter sets were used to reject backscattered and reflected excitation light. Images were acquired with V++ software (Total Turnkey Solutions, Sydney, Australia). The integration time for fluorescence images was 200 ms; 300-900 images were collected per lateral side per rat for lymph propulsive frequency measurements. Images were collected at or before day zero (when the first CIA injection was administered) and at days 11, 13, and 18 following CIA induction.

Results

As shown in FIGS. 5B-5G administration of etanercept using the Sofusa™ drug delivery platform decreased joint swelling over time in the CIA RA model in vivo. Surprisingly, the lymphatic pumping rate (i.e., the rate in which lymph is moved in the lymphatic system) correlated with the decrease in joint swelling in the CIA RA rat model.

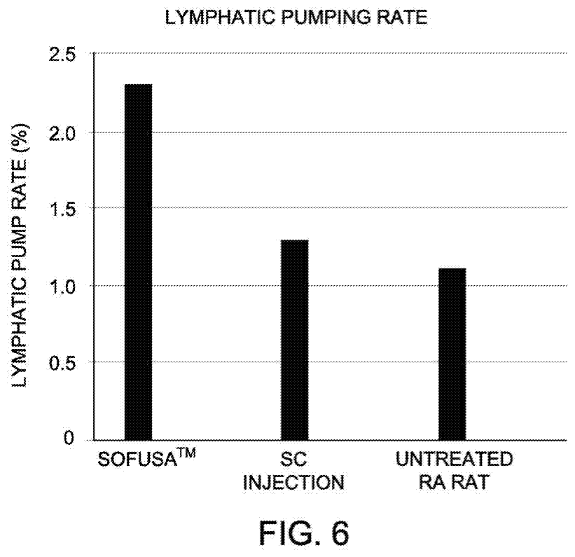

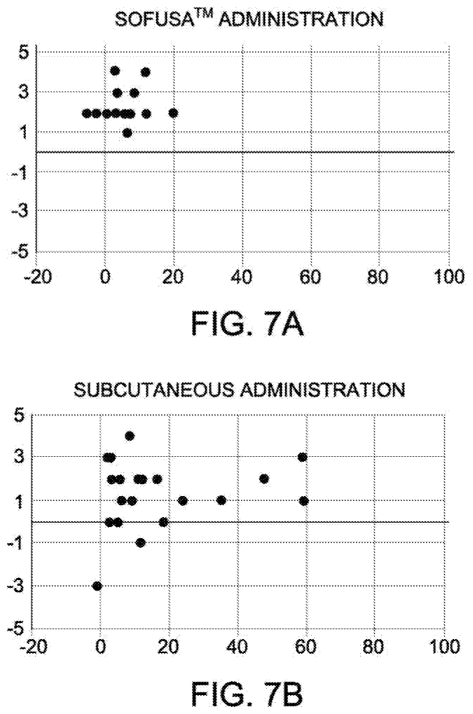

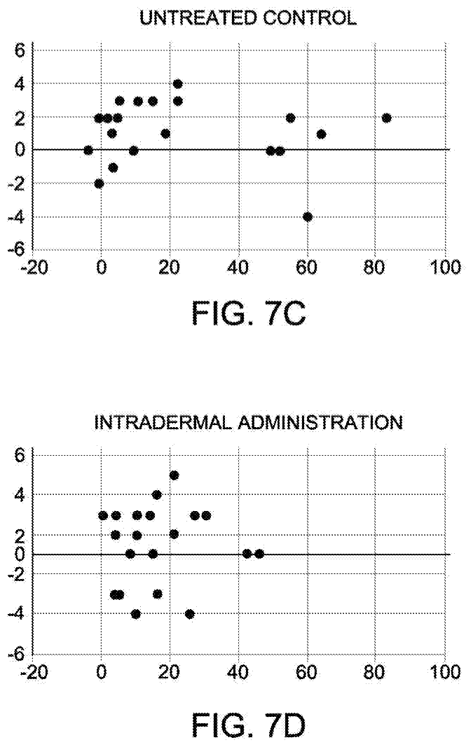

As shown in FIGS. 6 and 7A to 7D, administration of etanercept via the Sofusa™ drug delivery platform significantly increased the lymphatic pumping rate as compared to untreated, ID, and SC groups which was shown to correlate with reduced joint swelling and improved joint function in the CIA RA rat model.

At days 13 and 18 post CIA induction, univariate analyses showed: (1) statistically significant greater lymphatic pumping in the hind limbs of animals treated with SOFUSA™ and SC administrations than in untreated animals and (2) significant reduction in swelling of the hind limbs of all treated groups when compared to untreated animals. Animals treated by SOFUSA™ had significantly reduced hind limb swelling at days 13 and 18 when compared to SC-treated animals.

Paired group comparisons using contrast in the ANOVA model showed that no statistically significant increase in swelling was observed in animals treated by SOFUSA™ administration while swelling was significantly increased with progression of disease in untreated animals and animals treated by SC and ID administration.

Results in Yorkshire Pig Model

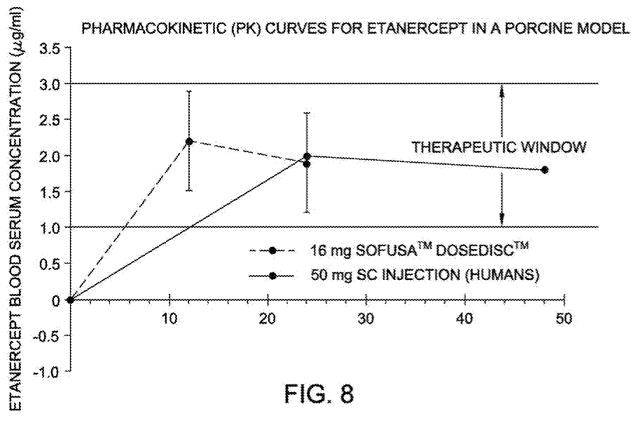

Significantly, the improved PK/PD profile initially observed in the rat model carried over to a porcine model. As shown in FIG. 8, administration of etanercept using the Sofusa™ drug delivery platform in a porcine model maintained a serum concentration at a therapeutically effective concentration at a dose in a porcine model that was much lower than when compared to subcutaneous administration. The use of a much lower dose of etanercept has the potential to reduce the side effects and possible adverse consequences associated therewith when compared to other routes of administration.

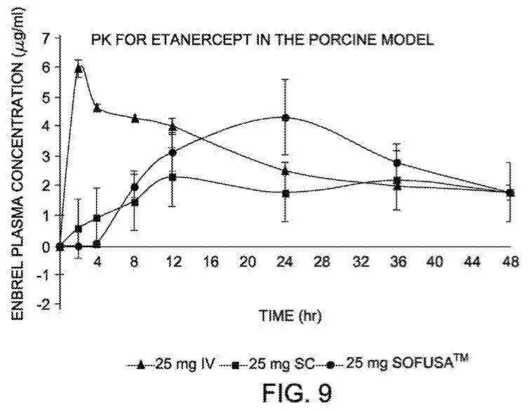

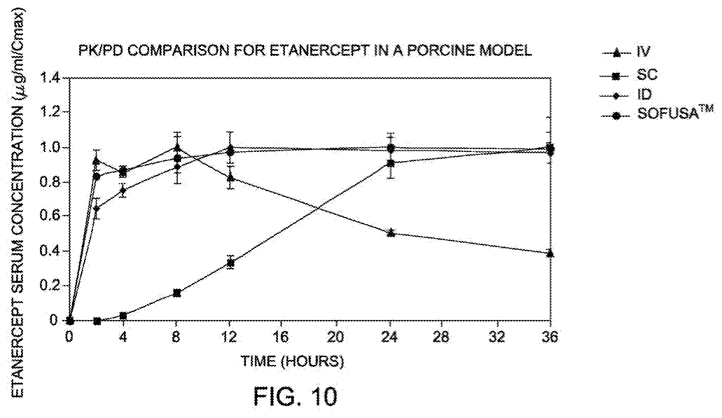

Shown in FIGS. 9 and 10, and in Table 4, is a comparison of the pharmacokinetic profile of etanercept in the porcine model when administered intravenously, subcutaneously and with the Sofusa™ drug delivery platform. The AUC₄₈ and bioavailability of etanercept was comparable to that for intravenous administration and significantly higher than that for subcutaneous delivery.

TABLE 4
Pharmacokinetic data for etanercept for three different
routes of administration in a porcine model.

| Delivery | Tₘₐₓ | Cₘₐₓ | AUC₀₋₄₈ | BA |
|---|---|---|---|---|
| Technology | (hrs) | (μg/mL) | (hrs-μg/mL) | (%) |
| IV | 2 | 6 | 143 | 100 |
| SC | 24 | 2 | 89 | 62 |
| Sofusa ™ | 24 | 4.4 | 132 | 93 |

### Example 2—Tumor Metastasis Study

Female BALB/c mice (15-20 g) were inoculated in the right mammary fat pad with 20K 4T1-luc cells (mouse mammary carcinoma). On days 11, 15, 19 and 23 post-inoculation, the mice were treated with 10 mg/kg anti-mCTLA-4 monotherapy (BioXcell clone 9H10) administered using the Sofusa™ drug delivery platform at a flow rate of 100 μL/hr. Delivery of the monotherapy was to the axillary lymph nodes on the same side of the rat as the tumor. Tumor volume was monitored periodically using calipers, and on day 30, the animals were sacrificed and bioluminescence imaging was done to determine the metastatic burden on each animal.

Results

Show in FIG. 12 is a graph of tumor volume over time for the rats treated using the Sofusa™ drug delivery platform using the anti-mCTLA-4 monotherapy as compared to both IP administration and an untreated control. As expected, the untreated control tumors were larger than those treated with the anti-mCTLA-4 monotherapy. However, tumor volume in the Sofusa™ treated animals was significantly lower than those treated by IP administration.

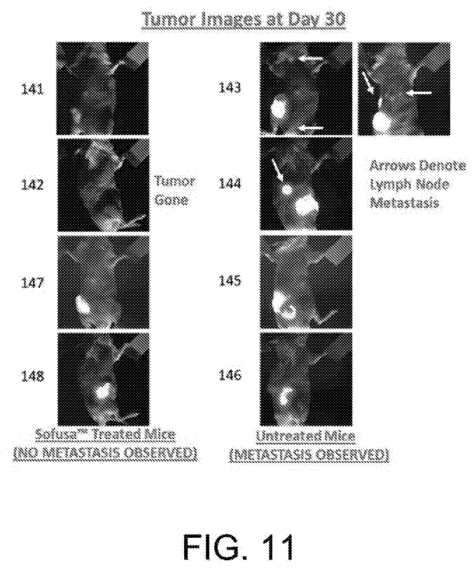

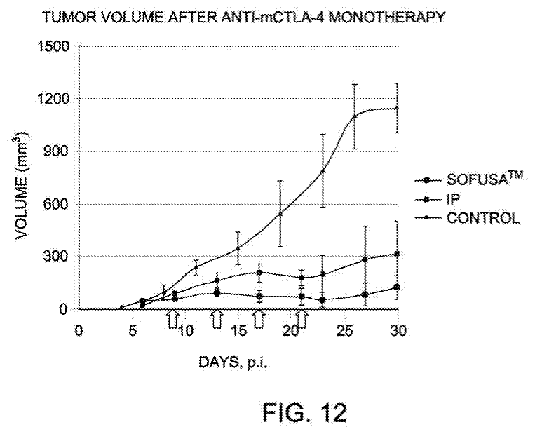

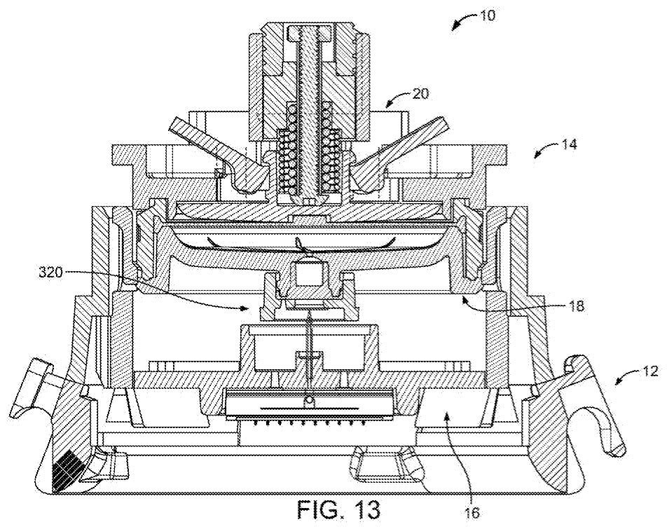

Additionally, as shown in FIG. 11, for the control animals (Sofusa™ administration of vehicle only; N=4) extensive metastasis was observed in all animals. In contrast, in animals treated with the anti-mCTLA-4 monotherapy (N=4), no metastasis was observed, and the tumor was either significantly reduced in size or entirely eliminated.

This written description uses examples to disclose the subject matter herein, including the best mode, and also to enable any person skilled in the art to practice the subject matter this disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for administering a therapeutic agent to the lymphatic system of a patient, the method comprising:
placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography;
placing a second medical device comprising a plurality of microneedles on the skin of the patient at a second location proximate to a second position under the skin of the patient, optionally wherein the first and second medical devices are the same device, wherein the second position is proximate to lymph vessels and/or lymph capillaries that drain into the thoracic duct, and wherein the microneedles of the second medical device have a surface comprising nanotopography;
inserting the plurality of microneedles of the first medical device into the patient to a depth whereby at least the epidermis is penetrated and an end of at least one of the microneedles is proximate to the first position;
inserting the plurality of microneedles of the second medical device into the patient to a depth whereby at least the epidermis is penetrated and an end of at least one of the microneedles is proximate to the second position; and
administering via the microneedles of the first medical device a first dose of the therapeutic agent into the first position; and administering via the microneedles of the second medical device a second dose of the therapeutic agent into a second position.

2. The method according to claim 1, wherein administering the first dose and administering the second dose is simultaneous.

3. The method according to any one of claim 1 or 2, wherein administering the first dose and administering the second dose partially overlap in time.

4. The method according to any one of claims 1-3, wherein administering the first dose and administering the second dose is sequential.

5. The method according to any one of claims 1-4, wherein the first and second medical devices are different devices.

6. The method according to claim 4, wherein the first and second medical devices are the same device.

7. The method according to any one of claims 1-6, wherein administering the doses cumulatively provides a therapeutically effective amount of the therapeutic agent.

8. The method according to any one of claims 1-7, wherein the first location and the second location are on different limbs of the patient.

9. The method according to any one of claims 1-8, wherein the first location and the second location are each independently proximate to the hands or the feet of the patient.

10. The method according to any one of claims 1-9, wherein one of the first location or the second location is on the right arm or the right leg of the patient and the other location is on the left arm or the left leg of the patient.

11. The method according to any one of claims 1-10, wherein the method further comprises:
placing a third medical device comprising a plurality of microneedles on the skin of the patient at a third location proximate to a third position under the skin of the patient, wherein the third position is proximate to lymph vessels and/or lymph capillaries;
inserting the plurality of microneedles of the third medical device into the patient to a depth whereby at least the epidermis is penetrated and an end of at least one of the microneedles is proximate to the third position; and
administering via the third medical device a third dose of said therapeutic agent; and
wherein the third location is different than the first location and the second location, and
the third position is different that the first position and the second position.

12. The method according to claim 11 wherein the first location, the second location and the third location are on different limbs of the patient.

13. The method according to claim 11 or 12,
wherein the first position, the second position and the third position are selected such that the therapeutic agent is administered to regions of the lymphatic system that initially drain into different lymph nodes, and
wherein the draining lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes.

14. The method according to any of claims 11-13, wherein the method further comprises:
placing a fourth medical device comprising a plurality of microneedles on the skin of the patient at a fourth location proximate to a fourth position under the skin of the patient, wherein the fourth position is proximate to lymph vessels and/or lymph capillaries;
inserting the plurality of microneedles of the fourth medical device into the patient to a depth whereby at least the epidermis is penetrated and an end of at least one of the microneedles is proximate to the fourth position; and
administering via the fourth medical device a fourth dose of said therapeutic agent; and
wherein the first location, the second location, the third location, and the fourth location are on different limbs of the patient.

15. The method according to claim 14, wherein the first dose and the second dose are administered simultaneously, and the third dose and the fourth dose are administered simultaneously, and
a beginning time for administering the first dose and the second dose is different than a beginning time for administering the third dose and the fourth dose with a period of time between the beginning times for administrating the doses.

16. The method according to any of claim 14 or 15, wherein
the third position drains into the right lymphatic duct; and
the fourth position drains into the thoracic duct.

17. The method of claim 16 wherein the first location and the third location on the skin of the patient are different from each other, and the first position and the third position are different from each other, and
the first position and the third position are selected such that the therapeutic agent is administered to regions of the lymphatic system that initially drain into different lymph nodes.

18. The method of claim 17, wherein the second location and the fourth location on the skin of the patient are different from each other, and the second portion and the fourth portion of the lymphatic system are different from each other, and
the second position and the fourth position are selected such that the therapeutic agent is administered to regions of the lymphatic system that initially drain into different lymph nodes.

19. The method according to any one of claims 1-18, wherein administering the first dose of the therapeutic agent and administering the second dose of the therapeutic agent partially overlap in time.

20. A method for administering a therapeutic agent to the lymphatic system of a patient, the method comprising:
placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography;
placing a second medical device comprising a plurality of microneedles on the skin of the patient at a second location proximate to a second position under the skin of the patient, wherein the second position is proximate to lymph vessels and/or lymph capillaries that drain into the thoracic duct, and wherein the microneedles of the second medical device have a surface comprising nanotopography, optionally wherein the first and second medical devices are the same medical device;
inserting the plurality of microneedles of the first medical device into the patient to a depth whereby at least the epidermis is penetrated and an end of at least one of the microneedles is proximate to the first position;
inserting the plurality of microneedles of the second medical device into the patient to a depth whereby at least the epidermis is penetrated and an end of at least one of the microneedles is proximate to the second position;
administering via the microneedles of the first medical device a first dose of the therapeutic agent into the first position; and
administering via the microneedles of the second medical device a second dose of the therapeutic agent into the second position,
wherein a beginning time for administering the first dose and the second dose are different and separated by a period of time.

21. The method according to claim 20, wherein the period of time is at least 4, 6, 8, 10, 12, 16, 24, 36, 48 or 72 hours.

22. The method according to any one of claim 20 or 21, wherein the first dose, the second dose, or the first and second doses together constitute a therapeutically effective dose.

23. The method according to any one of claims 20-22, wherein the first dose and the second dose are therapeutically effective doses.

24. The method according to claims 20-23, wherein the method further comprises:
placing a third medical device comprising a plurality of microneedles on the skin of the patient at a third location proximate to a third position under the skin of the patient, wherein the third position is proximate to lymph vessels and/or lymph capillaries, and wherein the microneedles of the third medical device have a surface comprising nanotopography;
inserting the plurality of microneedles of the third medical device into the patient to a depth whereby at least the epidermis is penetrated and an end of at least one of the microneedles is proximate to the third position; and
administering via the microneedles of the third medical device a third dose of the therapeutic agent into the third position;
wherein the beginning time for administering the first dose, the second dose, and the third dose are each separated by a period of time; and
the first location, the second location, and the third location are located on different limbs of the patient.

25. The method according to claims 20-23, wherein the method further comprises:
placing a third medical device comprising a plurality of microneedles on the skin of the patient at a third location proximate to a third position under the skin of the patient, wherein the third position is proximate to lymph vessels and/or lymph capillaries, and wherein the microneedles of the third medical device have a surface comprising nanotopography;
inserting the plurality of microneedles of the third medical device into the patient to a depth whereby at least the epidermis is penetrated and an end of at least one of the microneedles is proximate to the third position; and
administering via the microneedles of the third medical device a third dose of the therapeutic agent into the third position;
wherein the beginning time for administering the first dose, the second dose, and the effective dose are each separated by a period of time; and
wherein the first location and the third location are different and are selected such that the therapeutic agent is administered to regions of the lymphatic system that initially drain into different lymph nodes.

26. The method according to any one of claim 24 or 25, wherein the first dose, the second dose, the third dose, or a combination of any two or more thereof constitute a therapeutically effective dose.

27. The method according to any one of claims 24-26, wherein the first dose, the second dose, and the third dose are therapeutically effective doses.

28. The method according to any of claims 24-27, wherein the first position and the third position flow initially into different lymph nodes.

29. The method according to any of claims 24-28, wherein the method further comprises:
placing a fourth medical device comprising a plurality of microneedles on the skin of the patient at a fourth location proximate to a fourth position under the skin of the patient, wherein the fourth position is proximate to lymph vessels and/or lymph capillaries, and wherein the microneedles of the fourth medical device have a surface comprising nanotopography;
inserting the plurality of microneedles of the fourth medical device into the patient to a depth whereby at least the epidermis is penetrated and an end of at least one of the microneedles is proximate to the fourth position; and
administering via the microneedles of the fourth medical device a fourth therapeutically effective dose of the therapeutic agent into the fourth position; and
wherein the beginning time for administering the first dose, the second dose, the third dose, and the fourth dose are each separated by a period of time; and
wherein the lymph vessels and/or lymph capillaries of the third position drain into right lymphatic duct, and the lymph vessels and/or lymph capillaries of the fourth position drains into the thoracic duct,
wherein the first location and the third location are different and are selected such that the therapeutic agent is administered to regions of the lymphatic system that initially drain into different lymph nodes, and
wherein the second location and the fourth location are different and selected such that the therapeutic agent is administered to regions of the lymphatic system that initially drain into different lymph nodes.

30. The method according to claim 29, wherein the first dose, the second dose, the third dose, the fourth dose, or a combination of any two or more thereof constitute a therapeutically effective dose.

31. The method according to claim 29 or 30, wherein the first dose, the second dose, the third dose, and the fourth dose are therapeutically effective doses.

32. The method according to any one of claim 1-31, wherein the first and second locations deliver to lymphatic capillaries and/or vessels that drain into different lymph nodes.

33. The method according to any one of claims 1-32, wherein the lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

34. The method of any one of claims 1-33, wherein the first location is a first arm and the second location is selected from a leg or arm on the opposite of the body of the patient.

35. The method of any one of claims 1-34, wherein the therapeutic agent is an immune-suppressing agent.

36. The method of any one of claims 1-35, wherein the therapeutic agent is adalimumab, adalimumab-atto, certolizumab pegol, etanercept, etanercept-szzs, golimumab, infliximab, infliximab-dyyb, ustekinumab, rituximab, secukinumab, omalizumab, natalizumab, ixekizumab, obinutuzumab, rituximab/hyaluronidase human, dor a biosimilar or bioequivalent of any of the foregoing.

37. The method of any one of claims 1-36, wherein the therapeutic agent is an anti-CTLA-4 antibody.

38. A method for preventing or reducing cancer metastasis in a patient, the method comprising:
locating at least one lymph node in the patient that intervenes in the lymphatic system between a solid cancer tumor and a draining duct;
placing a medical device comprising a plurality of microneedles on the skin of the patient proximate to a first position under the skin of the patient located between the intervening lymph node and the solid cancer tumor, wherein the first position is proximate to lymph vessels and/or lymph capillaries in the patient's lymphatic system, and wherein the microneedles have a surface comprising nanotopography;
inserting the plurality of microneedles into the patient to a depth whereby at least the epidermis is penetrated and an end of at least one of the microneedles is proximate to the first position; and
administering via the plurality of microneedles to the first position a therapeutically effective amount of an anti-CTLA-4 antibody that is effective for preventing or reducing metastasis of the solid cancer tumor.

39. A method for preventing or reducing cancer metastasis in a patient, the method comprising:
locating a solid cancer tumor in the patient; locating at least one lymph node in the patient that intervenes in the lymphatic system between the solid cancer tumor and a draining duct;
placing a medical device that comprises a plurality of microneedles on the skin of the patient at a first location on the skin of the patient that is proximate to lymph capillaries and/or lymph vessels that flow into the intervening lymph node, wherein the microneedles have a surface comprising nanotopography;
inserting the plurality of microneedles into the patient to a depth whereby at least the epidermis is penetrated; and
administering via the plurality of microneedles to the lymph capillaries and/or lymph vessels that flow into the intervening lymph node a therapeutically effective amount of an anti-CTLA-4 antibody that is effective in preventing or reducing cancer metastasis.

40. A method of treating cancer in a patient, comprising:
placing a medical device comprising a plurality of microneedles on the skin of the patient proximate to a first position under the skin of the patient, wherein the first position is proximate to lymph vessels and/or lymph capillaries in the patient's lymphatic system, and wherein the microneedles have a surface comprising nanotopography;
inserting the plurality of microneedles into the patient to a depth whereby at least the epidermis is penetrated and an end of at least one of the microneedles is proximate to the first position; and
administering via the plurality of microneedles to the first position an anti-CTLA-4 antibody, thereby treating the cancer.

41. The method according to any one of claims 38-40, wherein the cancer comprises a tumor.

42. The method according to any one of claims 38-41, wherein the medical device is placed, relative to the tumor, distal to the draining duct.

43. The method according to claim 38-42, wherein at least one lymph node in the patient intervenes in the lymphatic system between the tumor and a draining duct; and the first position is located between the intervening lymph node and the tumor.

44. The method according to any one of claims 38-43, wherein the medical device is placed at a location on the skin of the patient having lymphatic capillaries and/or vessels that flow directly into the intervening lymph node without first passing through any prior lymph node.

45. The method according to any one of claims 38-44, wherein the cancer is a cancer of the head and neck, and the lymph nodes are selected from the group consisting of the jugular lymph nodes, the cervical lymph nodes, the supraclavicular lymph nodes, and combinations thereof.

46. The method according to any one of claims 38-44, wherein the cancer is an oral cavity cancer, and the lymph nodes are selected from the group consisting of the jugular lymph node chain, the cervical lymph nodes, the supraclavicular lymph nodes, and combinations thereof.

47. The method according to any one of claims 38-44, wherein the cancer is a cancer of the pharynx, and the lymph nodes are selected from the group consisting of the jugular lymph node chain, the cervical lymph nodes, the supraclavicular lymph nodes, and combinations thereof.

48. The method according to any one of claims 38-44, wherein the cancer is a melanoma, and the lymph nodes are selected from the group consisting of axillary lymph nodes, inguinal lymph nodes, jugular lymph nodes, cervical lymph nodes, supraclavicular lymph nodes, and combinations thereof.

49. The method according to any one of claims 38-44, wherein the cancer is breast cancer, and lymph nodes are selected from the group consisting of the axillary lymph nodes, the internal mammary lymph nodes, the supraclavicular lymph nodes and combinations thereof.

50. The method according to any one of claims 38-44, wherein the cancer is prostate cancer, and the lymph nodes are selected from the group consisting of the lumbar lymph nodes, the inguinal lymph nodes, the peritoneal lymph nodes and combinations thereof.

51. The method according to any one of claims 38-44, wherein the cancer is in the genital system of the patient with the proviso that it is not ovarian cancer, and the lymph nodes are selected from the group consisting of the lumbar lymph nodes, the inguinal lymph nodes, the peritoneal lymph nodes and combinations thereof.

52. The method according to any one of claims 38-51, wherein the anti-CTLA-4 antibody is imilimumab, a biosimilar thereof, or a bioequivalent thereof.

53. A method for treating an inflammatory medical condition in a patient, the method comprising:
locating at least one inflammatory locus in the patient, wherein the at least one inflammatory locus comprises lymph vessels, lymph capillaries, lymph nodes, lymph organs or any combination thereof;
locating a first position in the lymphatic system of the patient that is upstream of the inflammatory locus;
placing a medical device comprising a plurality of microneedles on the skin of the patient proximate to the first position, wherein the microneedles have a surface comprising nanotopography;
inserting the plurality of microneedles into the patient to a depth whereby at least the epidermis is penetrated; and
administering via the plurality of microneedles to the first position a therapeutically effective amount of an immune-suppressing agent that is effective in treating the inflammatory medical condition.

54. The method according to claim 53, wherein the upstream position in the lymphatic system is a lymph node selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

55. The method according to any one of claim 53 or 54, wherein the at least one inflammatory locus in the patient is a joint or a psoriatic lesion.

56. The method according to any one of claims 53-55, wherein the at least one inflammatory locus in the patient is a at least one joint selected from the group consisting of an ankle joint, a knee joint, a hip joint, a shoulder joint, an elbow joint, a metacarpophalangeal joint of the hands, a metatarsophalangeal joint in a foot, a wrist joint, a joint in the neck, and combinations thereof.

57. The method according to any one of claims 53-56, wherein the at least one inflammatory locus in the patient is a psoriatic lesion.

58. The method according to any one of claims 53-57, wherein the inflammatory medical condition is selected from the group consisting of Behcet's disease, sarcoidosis, rheumatoid arthritis (RA), juvenile arthritis, psoriatic arthritis, plaque psoriasis, hidradenitis suppurativa, non-infectious uveitis, ankylosing spondylitis, ulcerative colitis (UC), Crohn's disease, and combinations thereof.

59. A method for lowering the TNF-α level in a patient, the method comprising:
locating a first position in the lymphatic system of the patient;
placing a medical device comprising a plurality of microneedles on the skin of the patient proximate to the first position, and wherein the microneedles have a surface comprising nanotopography;
inserting the plurality of microneedles into the patient to a depth whereby at least the epidermis is penetrated; and
administering via the plurality of microneedles to the first position a therapeutically effective amount of an immune-suppressing agent that is effective in lowering the TNF-α level in the patient.

60. The method according to claim 59, wherein the first position is at least one lymph node of the patient.

61. A method for treating an inflammatory medical condition in a patient, the method comprising:
locating at least one inflammatory locus in the patient comprising lymph nodes, lymph capillaries, lymph vessel, lymph organs or any combination thereof;
placing a medical device comprising a plurality of microneedles on the skin of the patient proximate to a first position under the skin of the patient, wherein the first position is situated such that it comprises selected lymph capillaries and/or lymph vessels that deliver lymph directly into the lymphatic system in the inflammatory locus, and wherein the microneedles have a surface comprising nanotopography;
inserting the plurality of microneedles into the patient to a depth whereby at least the epidermis is penetrated; and
administering via the plurality of microneedles to the selected lymph capillaries and/or lymph vessels of the patient a therapeutically effective amount of an immune-suppressing agent that is effective in treating the inflammatory medical condition.

62. A method for treating an inflammatory medical condition in a patient, the method comprising:
placing a medical device comprising a plurality of microneedles on the skin of the patient proximate to a first position under the skin of the patient, wherein the first position is situated such that it comprises lymph capillaries and/or lymph vessels that deliver lymph directly into the lymphatic system, and wherein the microneedles have a surface comprising nanotopography;
inserting the plurality of microneedles into the patient to a depth whereby at least the epidermis is penetrated; and
administering via the plurality of microneedles to the lymph capillaries and/or lymph vessels of the patient an immune-suppressing agent, thereby treating the inflammatory medical condition.

63. The method according to any one of claims 53-62, wherein the first position is situated such that it comprises selected lymph capillaries and/or lymph vessels that deliver lymph directly into the lymphatic system in an inflammatory locus in the patient comprising lymph nodes, lymph capillaries, lymph vessel, lymph organs or any combination thereof.

64. The method according to any one of claims 53-63, wherein, relative to the inflammatory locus, the selected lymph capillaries and/or vessels are located distal to the heart of the patient.

65. The method according to any one of claims 53-64, wherein the at least one inflammatory locus in the patient is a joint.

66. The method according to any one of claims 53-65, wherein the at least one inflammatory locus in the patient is at least one joint selected from the group consisting of an ankle joint, a knee joint, a hip joint, a shoulder joint, an elbow joint, a metacarpophalangeal joint of the hands, a metatarsophalangeal joint in a foot, a wrist joint, a joint in the neck, and combinations thereof.

67. The method according to any one of claims 53-66, wherein the inflammatory locus is a knee, and the selected lymph capillaries and/or vessels flow into the popliteal lymph nodes.

68. The method according to any one of claims 53-66, wherein the inflammatory locus is a knee, and relative to the knee, the selected lymph capillaries and/or vessels are located distal to the heart.

69. The method according to any one of claims 53-66, wherein the inflammatory locus is the neck, and the selected lymph capillaries and/or vessels flow into the cervical lymph nodes.

70. The method according to any one of claims 53-66, wherein the inflammatory locus is the neck, and, relative to the neck, the selected lymph capillaries and/or vessels are located distal to the heart.

71. The method according to any one of claims 53-66, wherein the inflammatory locus is a shoulder, and the selected lymph capillaries and/or vessels flow into the pectoral lymph nodes, the superclavical lymph nodes, the axillary lymph nodes or any combination thereof.

72. The method according to any one of claims 53-66, wherein the inflammatory locus is a shoulder, and, relative to the shoulder, the selected lymph capillaries and/or vessels are located distal to the heart.

73. The method according to any one of claims 53-66, wherein the inflammatory locus is an elbow, and the selected lymph capillaries and/or vessels flow into the epitrochlear lymph nodes and/or brachial lymph nodes.

74. The method according to any one of claims 53-66, wherein the inflammatory locus is an elbow, and relative to the elbow, the selected lymph capillaries and/or vessels are located distal to the heart.

75. The method according to any one of claims 53-66, wherein the inflammatory locus is a hip, and the selected lymph capillaries and/or vessels flow into the inguinal lymph nodes and/or the pelvic lymph nodes.

76. The method according to any one of claims 53-66, wherein the inflammatory locus is a hip, and, relative to the hip, the selected lymph capillaries and/or vessels are located distal to the heart.

77. The method according to any one of claims 53-66, wherein the inflammatory locus is a hip, and, relative to the hip, the selected lymph capillaries and/or vessels are located proximate to the heart.

78. The method according to any one of claims 53-77, wherein the inflammatory medical condition is rheumatoid arthritis.

79. The method according to any one of claims 53-78, wherein the inflammatory locus is a psoriatic lesion.

80. The method according to claim 79, wherein the selected lymph capillaries share common lymph vessels and/or lymph capillaries immediately adjacent to and/or within the psoriatic lesion.

81. The method according to claim 79 or 80, wherein the medical device is placed at a location on the skin of the patient having lymph capillaries and/or vessels that flow directly into the lymph nodes within and/or closest to the psoriatic lesion.

82. The method according to claim 81, wherein, relative to the inflammatory locus, the first medical device administers a first therapeutic agent to selected lymph capillaries and/or vessels distal to the heart, and
the method further comprises administering via a second medical device a second therapeutic agent, which is an immune-suppressing agent, to selected lymph capillaries and/or vessels proximate to the heart.

83. The method according to claim 82, wherein the antibody that inhibits TNF-α or etanercept or a biosimilar or bioequivalent thereof administered to the first position and the second therapeutic agent are the same or different.

84. The method according to any one of claims 53-83, wherein the immune-suppressing agent is a TNF-α inhibitor.

85. The method according to any one of claims 53-83, wherein the immune-suppressing agent is adalimumab, adalimumab-atto, certolizumab pegol, etanercept, etanercept-szzs, golimumab, infliximab, infliximab-dyyb, ustekinumab, rituximab, secukinumab, omalizumab, natalizumab, ixekizumab, obinutuzumab, rituximab/hyaluronidase human, or a biosimilar or bioequivalent of any of the foregoing.

86. The method according to any one of claims 53-83, wherein the immune-suppressing agent is an antibody that inhibits TNF-α or etanercept or a biosimilar or bioequivalent thereof.

87. The method according to any one of claims 53-83, wherein the immune-suppressing agent is adalimumab or a biosimilar or bioequivalent thereof.

88. The method according to any one of claims 53-83, wherein the immune-suppressing agent is etanercept or a biosimilar or bioequivalent thereof.

89. The method according to claim 86, wherein the antibody that inhibits TNF-α or etanercept or a biosimilar or bioequivalent thereof is adalimumab or a biosimilar or bioequivalent thereof.

90. The method according to claim 86, wherein the antibody that inhibits TNF-α or etanercept or a biosimilar or bioequivalent thereof is etanercept or a biosimilar or bioequivalent thereof.

91. The method according to any one of claims 53-90, wherein the first medical device is placed at a location on the skin of the patient having lymph capillaries and/or vessels that flow directly into the lymph nodes within and/or closest to the inflammatory locus.

92. The method according to any one of claims 53-91, wherein the selected lymph capillaries and/or vessels, relative to the inflammatory locus, are distal to the heart.

93. The method according to any one of claims 53-92, wherein the selected lymph capillaries and/or vessels, relative to the inflammatory locus, are proximate to the heart.

94. The method according to any one of claims 1-93, wherein the patient is a mammal.

95. The method according to any one of claims 1-94, wherein the patient is a human.

96. The method according to any one of claims 1-95, wherein the medical device is a Sofusa™ drug delivery platform.

97. The method according to any one of claims 1-96, wherein the medical device comprises a fluid delivery apparatus,
wherein the fluid delivery apparatus comprises:
a fluid distribution assembly wherein a cap assembly is coupled to a cartridge assembly, and the cartridge assembly is slidably coupled to a plenum assembly, and a mechanical controller assembly is slidably coupled to the cartridge assembly;
a collet assembly constituting the housing of the fluid delivery apparatus and being slidably coupled to the fluid distribution assembly; and
a plurality of microneedles fluidically connected with the fluid distribution assembly having a surface comprising nanotopography, the plurality of microneedles being capable of penetrating the stratum corneum of the skin of a patient and controllably delivering the therapeutic agent, the anti-CTLA-4 antibody, or the immune-suppressing agent to a depth below the surface of the skin.

98. The method according to any one of claims 1-97, wherein the medical device delivers the therapeutic agent or the anti-CTLA-4 antibody or the immune-suppressing agent to a depth below the surface of the skin of from about 50 μm to about 4000 μm, from about 250 μm to about 2000 μm, or from about 350 μm to about 1000 μm.

99. The method according to any one of claims 1-98, wherein each of the microneedles in the medical device has a length between about 200 to about 800 μm, between about 250 to about 750 μm, or between about 300 to about 600 μm.
